SEQUENCE LISTING (20 sequences)

SEQ ID NO: 1 - protein, 21 aa, synthetic construct
  VARIANT 14: note = X= gamma-carboxyl-L-glutamic acid, L-alpha-aminoadipic acid, or 4-phosphono-L-phenylalanine
GIVEQCCTSICSLXQLENYCN

SEQ ID NO: 2 - protein, 29 aa, synthetic construct
  VARIANT 16: note = X= one N-3-methyl-L-histidine, 3-(3-pyridyl)-L-alanine, or 5-iodo-L-histidine
  VARIANT 25: note = X= N-3-methyl-L-histidine, 3-(3-pyridyl)-L-alanine, 2-Furyl-L-alanine, 2-thienyl-L-alanine, 4-thiazoyl-L-alanine, 3-pyridyl-L-alanine
  VARIANT 28: note = X= Proline, trans-4-hydroxy-L-proline, or (s)-piperidine-2-carboxylic acid
FVNQHLCGSHLVEALXLVCGERGFXYTXK

SEQ ID NO: 3 - protein, 21 aa, synthetic construct
  NON_STD 14: note = gamma-carboxyl-L-glutamic acid
GIVEQCCTSICSLXQLENYCN

SEQ ID NO: 4 - protein, 29 aa, synthetic construct
  NON_STD 16: note = N-3-methyl-L-histidine
  NON_STD 25: note = N-3-methyl-L-histidine
FVNQHLCGSHLVEALXLVCGERGFXYTPK

SEQ ID NO: 5 - skipped

SEQ ID NO: 6 - protein, 29 aa, synthetic construct
  NON_STD 16: note = 3-(3-pyridyl)-L-alanine
  NON_STD 25: note = 3-(3-pyridyl)-L-alanine
FVNQHLCGSHLVEALXLVCGERGFXYTPK

SEQ ID NO: 7 - skipped

SEQ ID NO: 8 - protein, 29 aa, synthetic construct
  NON_STD 16: note = 3-(3-pyridyl)-L-alanine
  NON_STD 25: note = 3-(3-pyridyl)-L-alanine
  NON_STD 28: note = trans-4-hydroxy-L-proline
FVNQHLCGSHLVEALXLVCGERGFXYTXK

SEQ ID NO: 9 - skipped

SEQ ID NO: 10 - protein, 29 aa, synthetic construct
  NON_STD 28: note = (s)-piperidine-2-carboxylic acid
  NON_STD 25: note = 3-(3-pyridyl)-L-alanine
  NON_STD 16: note = 3-(3-pyridyl)-L-alanine
FVNQHLCGSHLVEALXLVCGERGFXYTXK

SEQ ID NO: 11 - protein, 21 aa, synthetic construct
  NON_STD 14: note = L-alpha-aminoadipic acid
GIVEQCCTSICSLXQLENYCN

SEQ ID NO: 12 - protein, 29 aa, synthetic construct
  NON_STD 16: note = 5-iodo-L-histidine
  NON_STD 25: note = 2-Furyl-L-alanine
FVNQHLCGSHLVEALXLVCGERGFXYTPK

SEQ ID NO: 13 - protein, 21 aa, synthetic construct
  NON_STD 14: note = 4-phosphono-L-phenylalanine
GIVEQCCTSICSLXQLENYCN

SEQ ID NO: 14 - protein, 29 aa, synthetic construct
  NON_STD 16: note = 5-iodo-L-histidine
  NON_STD 25: note = 5-iodo-L-histidine
FVNQHLCGSHLVEALXLVCGERGFXYTPK

SEQ ID NO: 15 - skipped

SEQ ID NO: 16 - protein, 29 aa, synthetic construct
  NON_STD 16: note = 5-iodo-L-histidine
  NON_STD 25: note = 4-thiazoyl-L-alanine
FVNQHLCGSHLVEALXLVCGERGFXYTPK

SEQ ID NO: 17 - skipped

SEQ ID NO: 18 - protein, 29 aa, synthetic construct
  NON_STD 16: note = 5-iodo-L-histidine
  NON_STD 25: note = 3-pyridyl-L-alanine
FVNQHLCGSHLVEALXLVCGERGFXYTPK

SEQ ID NO: 19 - protein, 21 aa, synthetic construct
GIVEQCCTSICSLYQLENYCN

SEQ ID NO: 20 - protein, 29 aa, synthetic construct
FVNQHLCGSHLVEALYLVCGERGFFYTPK

(19) United States
(12) Patent Application Publication
(10) Pub. No.: US 2025/0154220 A1
(43) Pub. Date: May 15, 2025

(54) INSULIN ANALOGS FOR THE TREATMENT OF HUMAN METABOLIC DISORDER AND DISEASE

(71) Applicant: Ambio, Inc., Grovetown, GA (US)

(72) Inventors: Guoqing ZHANG, Shanghai (CN); Fuliang ZHANG, Shanghai (CN); Michael W. PENNINGTON, Sapphire, NC (US); Simrat P. SINGH, Evans, GA (US); Juncai BAI, Jacksonville, FL (US)

(21) Appl. No.: 18/945,043

(22) Filed: Nov. 12, 2024

(30) Foreign Application Priority Data
Nov. 15, 2023 (WO) PCT/CN2023/131773

(51) Int. Cl. C07K 14/62; A61K 38/00; A61P 3/10

(52) U.S. Cl. CPC C07K 14/62; A61P 3/10; A61K 38/00

(57) ABSTRACT

The disclosure described herein provides for compositions and uses of insulin analogs for stable and potent treatment of metabolic disease or disorder in a subject. In some embodiments, the insulin analog is a derivatized native insulin peptide (comprising an A-chain and B-chain) comprising 1, 2, 3, 4, 5, 6, 7, 8, 9, or 10 amino acid differences from native insulin. In typical embodiments, the insulin analogs described herein comprise an N-terminal, C-terminal, or both, substitution comprising a hydrophobic moiety. The insulin analogs described herein demonstrate potent receptor binding and extended plasma half-life. A pharmaceutical composition of the insulin analogs is described herein for the treatment of metabolic disease or disorder in a subject, such as diabetes.

### RELATED APPLICATIONS

This application claims priority to, and the benefit of, International Application PCT/CN2023/131773, filed on Nov. 15, 2023. The contents of which are incorporated herein by reference in its entirety.

### REFERENCE TO AN ELECTRONIC SEQUENCE LISTING

The contents of the electronic sequence listing (AMBO_002_001US_SeqList_ST26.xml; Size: 25,113 bytes; and Date of Creation: Nov. 5, 2024) are herein incorporated by reference in its entirety.

### BACKGROUND

Insulin is an important peptide hormone implicated in a range of diseases and disorders. It demonstrates the unparalleled ability to lower glucose in virtually all forms of diabetes. Insulin is a hormone secreted by the pancreas, which regulates blood glucose levels, and plays a role in maintaining normal blood glucose levels. In diabetic patients, however, insulin does not function properly due to lack of insulin, resistance to insulin, and loss of beta-cell function, and thus glucose in the blood cannot be utilized as an energy source and the blood glucose level is elevated, leading to hyperglycemia. Therefore, insulin therapy is essential for patients with abnormal insulin secretion (Type I) or insulin resistance (Type II), and blood glucose levels can be normally regulated by insulin administration. However, like other protein and peptide hormones, insulin has a very short in vivo half-life and thus has the disadvantage of repeated administration.

Additionally, insulin therapy is capable of excessive action that can lead to life-threatening hypoglycemia. Hypoglycemia in patients with diabetes mellitus is a frequent complication of insulin replacement therapy and can lead to significant morbidity (including altered mental status, loss of consciousness, seizures, and death). Indeed, fear of such complications poses a major barrier to efforts to obtain rigorous control of blood glucose concentrations and in patients with long-established Type 2 diabetes mellitus such efforts (“tight control”) may lead to increased mortality.

Accordingly, there is an urgent need for new diabetes treatment technologies that would reduce the risk of hypoglycemia while preventing upward increases of blood-glucose concentration above the normal range, while exhibiting a desirable half-life to limit overall injection frequency by the patient.

### SUMMARY OF THE INVENTION

The disclosure described herein are compositions and uses of insulin analogs for stable and potent treatment of metabolic disease or disorder in a subject. In some embodiments, the insulin analog is a derivatized native insulin peptide (comprising an A-chain and B-chain) comprising 1, 2, 3, 4, 5, 6, 7, 8, 9, or 10 amino acid differences from native insulin. In typical embodiments, the insulin analogs described herein comprise an N-terminal, C-terminal, or both, substitution comprising a hydrophobic moiety. The insulin analogs described herein demonstrate potent insulin receptor binding and extended plasma half-life. A pharmaceutical composition of the insulin analogs is described herein for the treatment of metabolic disease or disorder in a subject, such as diabetes. In some embodiments, the insulin analogs are administered to a human subject once weekly.

### BRIEF DESCRIPTION OF THE DRAWING(S)

FIGS. 1A-1M illustrate the structure of the insulin analogs of the present disclosure. FIG. 1A shows the structure of insulin analog 1. FIG. 1B shows the structure of insulin analog 2. FIG. 1C shows the structure of insulin analog 3. FIG. 1D shows the structure of insulin analog 4. FIG. 1E shows the structure of insulin analog Degludec. FIG. 1F shows the structure of insulin analog APi3543. FIG. 1G shows the structure of insulin analog APi3544. FIG. 1H shows the structure of insulin analog APi3545. FIG. 1I shows the structure of insulin analog APi3546. FIG. 1J shows the structure of insulin analog APi3547. FIG. 1K shows the structure of insulin analog APi3548. FIG. 1L shows the structure of insulin analog APi3549. FIG. 1M shows the structure of insulin analog APi3550.

FIG. 2 illustrates the glucose profile control of insulin analog #1 at various doses administered subcutaneously within a diabetic mouse model.

FIGS. 3A-3B illustrate the safety profile, as demonstrated by mean body weight (monitored over a three day period), of diabetic mice injected once with insulin analog #1 at various doses administered subcutaneously. FIG. 3A shows the safety profile as depicted in a line graph. FIG. 3B shows the safety profile as depicted in a bar graph.

FIG. 4 illustrates the calculated IU/mg (biopotency) for eight insulin analogs

FIG. 5 illustrates glucose levels in blood following administration in ICR mice.

FIG. 6 summarizes the calculated and experimentally measured in ICR mice against the insulin reference standard biopotency of the insulin analogs.

FIG. 7 illustrates the biopotency of insulin analogs (IU/mg) measured in ICR mice against the insulin reference standard.

FIGS. 8A-8C illustrates the glucose lowering effect of insulin analogs 2, 3, and 4 compared to Degludec. FIG. 8A shows the glucose levels (nmol/L) in rats injected with the insulin analogs compared to rats injected with Degludec over a period of 120 hours. FIG. 8B shows the normalized glucose levels (%) in rats injected with the insulin analogs compared to rats injected with Degludec over a period of 120 hours. FIG. 8C shows the glucose level delta (nmol/L) in rats injected with the insulin analogs compared to rats injected with Degludec over a period of 120 hours.

FIG. 9 illustrates the 0-96 hour glucose area under the curve (AUC) values of rats injected with insulin analogs 2, 3, and 4 compared to rats injected with Degludec.

FIG. 10 illustrates the normalized glucose levels (%) in rats injected with insulin analogs 2, 3, and 4 compared to rats injected with Degludec and Icodec over a period of 120 hours.

FIGS. 11A-11C illustrates the effect on c-peptide levels of insulin analogs 2, 3 and 4. FIG. 11A shows the c-peptide levels (ng/mL) in rats injected with the insulin analogs compared to rats injected with Degludec over a period of 120 hours. FIG. 11B shows the normalized c-peptide levels (%) in rats injected with the insulin analogs compared to rats injected with Degludec over a period of 120 hours. FIG. 11C shows the normalized c-peptide levels (%) in rats injected with the insulin analogs compared to rats injected with Degludec and Icodec over a period of 120 hours.

FIG. 12 illustrates HbA1C levels of rats injected with insulin analogs 2, 3, and 4 compared to rats injected with Degludec 96 hours post injection.

FIG. 13 illustrates the normalized body weight (%) in rats injected with insulin analogs 2, 3, and 4 compared to rats injected with Degludec over a period of 120 hours.

FIGS. 14A-14C illustrates the glucose lowering effect of insulin analogs compared to Degludec. FIG. 14A shows the glucose levels (nmol/L) in rats injected with insulin analogs 1, 2, 3, 4, APi3545, APi3546, APi3547, and APi3549 compared to rats injected with Degludec over a period of 120 hours. FIG. 14B shows the normalized glucose levels (%) in rats injected with insulin analogs 1, 2, 3, 4, APi3545, APi3546, APi3547, and APi3549 compared to rats injected with Degludec over a period of 120 hours. FIG. 14C shows the normalized glucose levels (%) in rats injected with insulin analogs 1, 2, 3, and 4 compared to rats injected with Degludec over a period of 120 hours.

FIG. 15 illustrates the 0-72 hour glucose area under the curve (AUC) values of rats injected with insulin analogs 2, 3, 4, APi3545, APi3546, APi3547, and APi3549 compared to rats injected with Degludec.

FIG. 16 illustrates the normalized body weight (%) in rats injected with insulin analogs 2, 3, 4, APi3545, APi3546, APi3547, and APi3549 compared to rats injected with Degludec over a period of 120 hours.

FIGS. 17A-17B illustrates the effect on c-peptide levels of the insulin analogs compared to Degludec. FIG. 17A shows the normalized c-peptide levels (%) in rats injected with insulin analogs 2, 3, 4, APi3545, APi3546, APi3547, and APi3549 compared to rats injected with Degludec over a period of 120 hours. FIG. 17B shows the normalized c-peptide levels (%) in rats injected with insulin analogs 2, 3, and 4 compared to rats injected with Degludec over a period of 120 hours.

FIG. 18 illustrates HbA1C levels of rats injected with insulin analogs 2, 3, 4, APi3545, APi3546, APi3547, and APi3549 compared to rats injected with Degludec 96 hours post injection.

FIG. 19 illustrates the lack of IGF-1R activation of insulin analogs 1, 2, 3, and 4 compared to IGF-1, insulin, and Degludec.

### DETAILED DESCRIPTION OF THE INVENTION

A simple system is used to describe fragments and analogs of insulin. In particular, insulin is comprised of a peptide A-chain and a peptide B-chain. Accordingly, A1 corresponds to the first N-terminal amino acid in the A-chain, with consecutive numbering following from N-terminus to C-terminus. For example, an A-chain comprising A1-A2-A3-A4-A5-A6-A7-A8-A9-A10-A11-A12-A13-A14-A15-A16-A17-A18-A19-A20-A21 is understood to comprise an amino acid A1 at the N-terminal end of the peptide chain and an amino acid A21 at the C-terminal end the peptide chain. Similarly, B1 corresponds to the first N-terminal amino acid in the B-chain, with consecutive numbering following from N-terminus to C-terminus. For example, a B-chain comprising B1-B2-B3-B4-B5-B6-B7-B8-B9-B10-B11-B12-B13-B14-B15-B16-B17-B18-B19-B20-B21-B22-B23-B24-B25-B26-B27-B28-B29 is understood to comprise an amino acid B1 at the N-terminal end of the peptide chain and an amino acid B29 at the C-terminal end the peptide chain.

The term “an analog” is defined herein as a peptide wherein one or more amino acid residues of the parent (or wild-type) insulin peptide (comprising an A-chain and a B-chain) have been substituted by another amino acid residue. In some embodiments, the substituted amino acid is a non-natural amino acid. In some embodiments, the substituted amino acid has the D-configuration.

In some embodiments, the insulin analog comprises at least one γ-carboxyl-L-glutamic acid (Gla). In some embodiments, the insulin analog comprises at least one N-3-methyl-L-histidine (His(3-Me)). In some embodiments, the insulin analog comprises at least one 3-(3-pyridyl)-L-alanine (Pal). In some embodiments, the insulin analog comprises at least one trans-4-hydroxy-L-proline (Hyp). In some embodiments, the insulin analog comprises at least one (s)-piperidine-2-carboxylic acid (Pip). In some embodiments, the insulin analog comprises at least one 2-Furyl-L-alanine (2Fal). In some embodiments, the insulin analog comprises at least one L-alpha-aminoadipic acid (Aad). In some embodiments, the insulin analog comprises at least one 5-iodo-L-histidine (His(5-I)). In some embodiments, the insulin analog comprises at least one 2-thienyl-L-alanine (Thi). In some embodiments, the insulin analog comprises at least one 4-phosphono-L-phenylalanine (Ppa). In some embodiments, the insulin analog comprises at least one 4-thiazoyl-L-alanine (Ala (4-thiazoyl)). In some embodiments, the insulin analog comprises at least one 3-pyridyl-L-alanine (3-Pal).

In certain embodiments, the analogs described herein comprise a non-natural or an un-natural amino acid mutation at any one of position A14, position B16, and position B25. In certain embodiments, the insulin analog further comprises one or more fatty acid substituents, such as one or more of a C20 (20 carbons) diacid. In some embodiments, the incorporation of a non-natural or un-natural amino acid mutation at position A14, position B16, and position B25 reduces analog enzymatic degradation by pepsin, chymotrypsin, or carboxypeptidase A, and increases its solubility and slow down receptor-mediated clearance.

In typical embodiments, the insulin analogs are peptide analogs. In typical embodiments, the insulin analogs are chemically synthesized. In typical embodiments, the insulin analogs described herein are not produced by recombinant technology.

In some embodiments, the analogs described herein demonstrate an extended half-life compared to wild type insulin or 1^{st} generation insulin products (animal insulin), or 2^{nd} generation insulin products (i.e., biosynthetic insulin such as Humulin or Novolin), or 3^{rd} generation insulin products (biosynthetic-made insulins such as Humalog or NovoRapid).

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 1); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Gla, or Aad, or Ppa, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 2); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(3-Me), or Pal, or His(5-I), B17 is Leu, B18 is Val, B19 is Cys, B20 is Gly, B21 is Glu, B22 is Arg, B23 is Gly, B24 is Phe, B25 is His(3-Me), or Pal, or 2Fal, or Thi, or Ala(4-thiazoyl), or 3-Pal, B26 is Tyr, B27 is Thr, B28 is Pro, or Hyp, or Pip, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 3); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Gla, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 4); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(3-Me), B17 is Leu, B18 is Val, B19 is Cys, B20 is Gly, B21 is Glu, B22 is Arg, B23 is Gly, B24 is Phe, B25 is His(3-Me), B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 3); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Gla, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 6); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is Pal, B18 is Val, B19 is Cys, B20 is Gly, B21 is Glu, B22 is Arg, B23 is Gly, B24 is Phe, B25 is Pal, B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 3); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Gla, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 8); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is Pal, B17 is Leu, B18 is Val, B19 is Cys, B20 is Gly, B21 is Glu, B22 is Arg, B23 is Gly, B24 is Phe, B25 is Pal, B26 is Tyr, B27 is Thr, B28 is Hyp, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 3); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Gla, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 10); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is Pal, B17 is Leu, B18 is Val, B19 is Cys, B20 is Gly, B21 is Glu, B22 is Arg, B23 is Gly, B24 is Phe, B25 is Pal, B26 is Tyr, B27 is Thr, B28 is Pip, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 11); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Aad, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 12); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is 2Fal, B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A1 l-A12-A13-A14-A15-A16-A17-A18-A19-A20-A21 (SEQ ID NO: 13); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Ppa, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 14); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is Thi, B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 11); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Aad, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 16); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is Ala(4-thiazoyl), B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 11); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Aad, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 18); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is 3-Pal, B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 11); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Aad, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 12); wherein BI is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is 2Fal, B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A1 l-A12-A13-A14-A15-A16-A17-A18-A19-A20-A21 (SEQ ID NO: 13); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Ppa, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 14); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is Thi, B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 11); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Aad, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 16); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is Ala(4-thiazoyl), B26 is Tyr, B27 is Thr, B28 is Pro, B29 is Lys.

In some embodiments, the insulin analog comprises a peptide A-chain and a peptide B-chain, wherein the A-chain from N-terminus to C-terminus comprises A1-A2-A3-A4-A5-A6-A7-A8-A9-A10-A11-A12-A13-A14-A15-A16-A17-A18-A19-A20-A21 (SEQ ID NO: 11); wherein A1 is Gly, A2 is Ile, A3 is Val, A4 is Glu, A5 is Gln, A6 is Cys, A7 is Cys, A8 is Thr, A9 is Ser, A10 is Ile, A11 is Cys, A12 is Ser, A13 is Leu, A14 is Aad, A15 is Gln, A16 is Leu, A17 is Glu, A18 is Asn, A19 is Tyr, A20 is Cys, A21 is Asn; and wherein the B-chain from N-terminus to C-terminus comprises B1-B2-B3-B4-B5-B6-B7-B8-B9-B10-B11-B12-B13-B14-B15-B16-B17-B18-B19-B20-B21-B22-B23-B24-B25-B26-B27-B28-B29 (SEQ ID NO: 18); wherein B1 is Phe, B2 is Val, B3 is Asn, B4 is Gln, B5 is His, B6 is Leu, B7 is Cys, B8 is Gly, B9 is Ser, B10 is His, B11 is Leu, B12 is Val, B13 is Glu, B14 is Ala, B15 is Leu, B16 is His(5-I), B17 is Leu, B18 is Val, B19 is Cys, B20 is Gly, B21 is Glu, B22 is Arg, B23 is Gly, B24 is Phe, B25 is 3-Pal, B26 is Tyr, B27 is Thr, B28 is Pro, B29 is Lys.

In some embodiments, the insulin analog comprises a peptide A-chain and a peptide B-chain, wherein the peptide A-chain comprises SEQ ID NO: 3 and the peptide B-chain comprises SEQ ID NO: 4. In some embodiments, the insulin analog that comprises a peptide A-chain and a peptide B-chain, wherein the peptide A-chain comprises SEQ ID NO: 3 and the peptide B-chain comprises SEQ ID NO: 4 is insulin analog 1.

In some embodiments, the insulin analog comprises a peptide A-chain and a peptide B-chain, wherein the peptide A-chain comprises SEQ ID NO: 3 and the peptide B-chain comprises SEQ ID NO: 6. In some embodiments, the insulin analog that comprises a peptide A-chain and a peptide B-chain, wherein the peptide A-chain comprises SEQ ID NO: 3 and the peptide B-chain comprises SEQ ID NO: 6 is insulin analog 2.

In some embodiments, the insulin analog comprises a peptide A-chain and a peptide B-chain, wherein the peptide A-chain comprises SEQ ID NO: 3 and the peptide B-chain comprises SEQ ID NO: 8. In some embodiments, the insulin analog that comprises a peptide A-chain and a peptide B-chain, wherein the peptide A-chain comprises SEQ ID NO: 3 and the peptide B-chain comprises SEQ ID NO: 8 is insulin analog 3.

In some embodiments, the insulin analog comprises a peptide A-chain and a peptide B-chain, wherein the peptide A-chain comprises SEQ ID NO: 3 and the peptide B-chain comprises SEQ ID NO: 10. In some embodiments, the insulin analog that comprises a peptide A-chain and a peptide B-chain, wherein the peptide A-chain comprises SEQ ID NO: 3 and the peptide B-chain comprises SEQ ID NO: 10 is insulin analog 4.

In some embodiments, the insulin analog comprises a peptide A-chain and a peptide B-chain, wherein the peptide A-chain comprises SEQ ID NO: 11 and the peptide B-chain comprises SEQ ID NO: 12. In some embodiments, the insulin analog that comprises a peptide A-chain and a peptide B-chain, wherein the peptide A-chain comprises SEQ ID NO: 11 and the peptide B-chain comprises SEQ ID NO: 12 is insulin analog APi3543. In some embodiments, the insulin analog that comprises a peptide A-chain and a peptide B-chain, wherein the peptide A-chain comprises SEQ ID NO: 11 and the peptide B-chain comprises SEQ ID NO: 12 is insulin analog APi3547.

In some embodiments, the insulin analog comprises a peptide A-chain and a peptide B-chain, wherein the peptide A-chain comprises SEQ ID NO: 13 and the peptide B-chain comprises SEQ ID NO: 14. In some embodiments, the insulin analog that comprises a peptide A-chain and a peptide B-chain, wherein the peptide A-chain comprises SEQ ID NO: 13 and the peptide B-chain comprises SEQ ID NO: 14 is insulin analog APi3544. In some embodiments, the insulin analog that comprises a peptide A-chain and a peptide B-chain, wherein the peptide A-chain comprises SEQ ID NO: 13 and the peptide B-chain comprises SEQ ID NO: 14 is insulin analog APi3548.

In some embodiments, the insulin analog comprises a peptide A-chain and a peptide B-chain, wherein the peptide A-chain comprises SEQ ID NO: 11 and the peptide B-chain comprises SEQ ID NO: 16. In some embodiments, the insulin analog that comprises a peptide A-chain and a peptide B-chain, wherein the peptide A-chain comprises SEQ ID NO: 11 and the peptide B-chain comprises SEQ ID NO: 16 is insulin analog APi3545. In some embodiments, the insulin analog that comprises a peptide A-chain and a peptide B-chain, wherein the peptide A-chain comprises SEQ ID NO: 11 and the peptide B-chain comprises SEQ ID NO: 16 is insulin analog APi3549.

In some embodiments, the insulin analog comprises a peptide A-chain and a peptide B-chain, wherein the peptide A-chain comprises SEQ ID NO: 11 and the peptide B-chain comprises SEQ ID NO: 18. In some embodiments, the insulin analog that comprises a peptide A-chain and a peptide B-chain, wherein the peptide A-chain comprises SEQ ID NO: 11 and the peptide B-chain comprises SEQ ID NO: 18 is insulin analog APi3546. In some embodiments, the insulin analog that comprises a peptide A-chain and a peptide B-chain, wherein the peptide A-chain comprises SEQ ID NO: 11 and the peptide B-chain comprises SEQ ID NO: 18 is insulin analog APi3550.

The disclosure herein also provides for insulin analogs that are substituted with a substituent. In some embodiments, the B-chain N-terminus or the B-chain C-terminus or both the N-terminus and C-terminus is substituted with a substituent. In some embodiment, the B-chain comprises a substituent attached to a B-chain lysine (Lys). In some embodiments, at least one lysine F-amino group of the insulin analog is substituted with a lipophilic substituent. In some embodiments, the F-amino group Lys of the insulin analog is substituted with a lipophilic substituent via a spacer.

In certain embodiments, the B-chain N-terminus (i.e., via the N-terminal nitrogen group on the B-peptide chain) or the B-chain C-terminus (i.e., at a c-terminal lysine) or both the N-terminus and C-terminus is substituted with a C20 diacid or a AEEA-AEEA-γ-Glu-(CH2)ₘ—OH (AEEA=2-[2-(2-aminoethoxy)ethoxy]acetic acid) wherein m is 12, 13, 14, 15, 16, 17, 18, 19, 20, 21, or 22. In certain embodiments, the B-chain N-terminus or the B-chain C-terminus or both the N-terminus and C-terminus is substituted with a AEEA-AEEA-γ-Glu-(CH2)ₘ—OH (AEEA=2-[2-(2-aminoethoxy)ethoxy]acetic acid) wherein m is 20.

In typical embodiments, the insulin analogs described herein comprise at least one linker. In certain embodiments, the A-chain comprises an intramolecular linker. In certain embodiments, the A-chain comprises an intermolecular linker. In certain embodiments, the B-chain comprises an intramolecular linker. In certain embodiments, the B-chain comprises an intermolecular linker. In some embodiments, A6 and A11 are connected by an intramolecular linker. In some embodiments, A7 and B7 are connected by an intermolecular linker. In some embodiments, A20 and B19 are connected by an intermolecular linker. In some embodiments, the linker is cleavable or non-cleavable. In certain embodiments, the linker is cleavable or liable. In certain embodiments, the linker is cleaved by pH-mediated cleavage. In some embodiments, the linker is cleaved by an enzymatic process. In some embodiments, the linker is cleaved by a chemical reduction. In typical embodiments the linker comprises at least a disulfide bond or bridge.

In some embodiments, the insulin analogs of the present disclosures exhibit an insulin analog binding affinity (EC₅₀) to insulin receptor or GLP-1 receptor in the nanomolar range. In some embodiments, the insulin analog binding affinity (EC₅₀) to insulin receptor or GLP-1 receptor of the insulin analog is less than 1000 nM, less than 900 nM, less than 800 nM, less than 700 nM, less than 600 nM, less than 500 nM, less than 400 nM, less than 300 nM, less than 200 nM. In some embodiments, the insulin analog binding affinity (EC₅₀) to insulin receptor or GLP-1 receptor is about 100 nM or less, e.g., about 75 nM or less, about 50 nM or less, about 25 nM or less, about 10 nM or less, about 5 nM or less, or about 1 nM or less. In some or any embodiments, the insulin analog of the present disclosures exhibits an EC₅₀ in the picomolar range. In exemplary embodiments, the insulin analog binding affinity (EC₅₀) to insulin receptor or GLP-1 receptor is less than 1000 pM, less than 900 pM, less than 800 pM, less than 700 pM, less than 600 pM, less than 500 pM, less than 400 pM, less than 300 pM, less than 200 pM. In some embodiments, the insulin analog binding affinity (EC₅₀) to insulin receptor or GLP-1 receptor is about 100 pM or less, e.g., about 75 pM or less, about 50 pM or less, about 25 pM or less, about 10 pM or less, about 5 pM or less, or about 1 pM or less.

In some embodiments, the insulin analog binding affinity (EC₅₀) to insulin receptor or GLP-1 receptor is below 10 nM. In some embodiments, the insulin analog binding affinity (EC₅₀) to insulin receptor is below 9 nM, or 8 nM, or 7 nM, or 6 nM. In some embodiments, the insulin analog binding affinity (EC₅₀) to insulin receptor is below 5 nM. In certain embodiments, the insulin analog binding affinity (EC₅₀) to insulin receptor is below 4 nM, or 3.5 nM, or 3 nM, or 2.5 nM, or 2 nM, or 1.5 nM. In certain embodiments, the insulin analog binding affinity (EC₅₀) to insulin receptor is below 1 nM. In certain embodiments, the insulin analog binding affinity (EC₅₀) to insulin receptor is below 0.9 nM, or 0.8 nM, or 0.7 nM, or 0.6 nM, or 0.5 nM, or 0.4 nM, or 0.3 nM, or 0.2 nM, or 0.1 nM. In certain embodiments, the insulin analog binding affinity (EC₅₀) to insulin receptor is below 0.09 nM, or 0.08 nM, or 0.07 nM, or 0.06 nm or 0.05 nM, or 0.04 nM, or 0.03 nM, or 0.02 nM, or 0.01 nM.

The present disclosure is directed to insulin analogs that retain high activity at the insulin receptor to native insulin, wherein the insulin analog exhibits 70%, 80%, 90%, 95%, 100% or greater activity at the insulin receptor relative to native insulin.

In some embodiments, the insulin analogs of the present invention comprise activity at both the insulin receptor or GLP-1 receptor. In some embodiments, the insulin analogs of the present invention comprise an extended duration of action at each of these receptors.

In some embodiments, the insulin analog binding affinity is measured by surface plasmon resonance or other similar methods of mass-dependent spectrometry. In some embodiments, the insulin analog binding affinity is measured by a gel-shift assay. In some embodiments, the insulin analog binding affinity is measured by a competition assay. In some embodiments, the insulin analog binding affinity is measured by an ELISA assay.

In some embodiments, the insulin analog binding is determined by imaging. In some embodiments, the insulin analog binding is determined by fluorescence imaging. In some embodiments, the insulin analog binding is determined by a radioligand binding assay.

In some embodiments, the insulin analog binding affinity is measured using a recombinant cell line. In some embodiments, the insulin analog binding affinity is measured using a mammalian recombinant cell line. In some embodiments, the insulin analog binding affinity is measured using a human recombinant cell line. In some embodiments, the insulin analog binding affinity is measured using a non-human recombinant cell line. In some embodiments, the recombinant cell line expresses a metabolic or hormone receptor.

In certain embodiments, the insulin analog binding affinity (EC₅₀) to the insulin receptor is determined by measuring luciferase activity using a recombinant cell reporter assay, system, or kit. In certain embodiments, the insulin analog binding affinity (EC₅₀) to the insulin receptor is determined by measuring luciferase activity using a recombinant cell reporter assay, wherein luciferase expression is under the control of a cAMP response element (CRE) and wherein the recombinant cell constitutively expresses insulin receptor.

### Compositions and Formulations

In certain embodiments, the insulin analog exhibits an extended in vivo plasma elimination half-life of at least 5 minutes, 10 minutes, 20 minutes, 30 minutes, 40 minutes, 50 minutes, or 60 minutes. In certain embodiments, the insulin analog exhibits an extended in vivo plasma elimination half-life of at least 1.5 hours, 2.0 hours, 2.5 hours, 3.5 hours, 4.0 hours, 4.5 hours, 5.0 hours, 5.5 hours, 6.0 hours, 6.5 hours, 7.0 hours, 7.5 hours, 8.0 hours, 8.5 hours, 9.0 hours, or 9.5 hours. In certain embodiments, the insulin analog exhibits an extended in vivo plasma elimination half-life of at least 10 hours, at least 15 hours, or at least 20 hours. In certain embodiments, the insulin analog exhibits an extended in vivo plasma elimination half-life of at least 24 hours. In certain embodiments, the insulin analog exhibits an extended in vivo plasma elimination half-life of at least one week. In certain embodiments, the insulin analog exhibits an extended in vivo plasma elimination half-life of at least 24 hours. In certain embodiments, the insulin analog exhibits an extended in vivo plasma elimination half-life of at least 168 hours. In certain embodiments, the insulin analog exhibits an extended in vivo plasma elimination half-life of at least 24 hours. In certain embodiments, the insulin analog exhibits an extended in vivo plasma elimination half-life of at least one week.

In some embodiments, the composition of the present invention can include liquid, solid, or semi-solid forms. Non-limiting examples of composition forms include liquid solutions, injectable solutions, infusible solutions, dispersions, suspensions, tablets, pills, powders, liposomes, suppositories, gels, or hydrogels.

In some embodiments, a given concentration of the insulin analog remains stable following storage for several days, weeks, months, or years at a given temperature. In some embodiments, the insulin analog remains stable for at least 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months, 2 years, 3 years, 4 years, or 5 years at a temperature that includes but is not limited to, at least −200° C., −100° C., −80° C., −70° C., −60° C., −50° C., −40° C., −30° C., −20° C., −10° C., −5° C., 1C, 2° C., 3C, 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C.

In some embodiments, the insulin analog of the present invention comprises a concentration of about 0.01 mg/kg to 0.05 mg/kg, 0.05 mg/kg to 0.10 mg/kg, 0.10 mg/kg to 0.15 mg/kg, 0.15 mg/kg to 0.20 mg/kg, 0.20 mg/kg to 0.25 mg/kg, 0.25 mg/kg to 0.30 mg/kg, 0.30 mg/kg to 0.35 mg/kg, 0.35 mg/kg to 0.40 mg/kg, 0.40 mg/kg to 0.45 mg/kg, 0.45 mg/kg to 0.50 mg/kg, 0.50 mg/kg to 0.55 mg/kg, 0.55 mg/kg to 0.60 mg/kg, 0.60 mg/kg to 0.65 mg/kg, 0.65 mg/kg to 0.70 mg/kg, 0.70 mg/kg to 0.75 mg/kg, 0.75 mg/kg to 0.80 mg/kg, 0.80 mg/kg to 0.85 mg/kg, 0.85 mg/kg to 0.90 mg/kg, 0.90 mg/kg to 0.95 mg/kg, 0.95 mg/kg to 1.00 mg/kg.

In some embodiments, the insulin analog of the present invention comprises a concentration of about 50 nmol/kg to 75 nmol/kg, 75 nmol/kg to 100 nmol/kg, 100 nmol/kg to 125 nmol/kg, 125 nmol/kg to 150 nmol/kg, 150 nmol/kg to 175 nmol/kg, 175 nmol/kg to 200 nmol/kg, 200 nmol/kg to 225 nmol/kg, 225 nmol/kg to 250 nmol/kg, 250 nmol/kg to 275 nmol/kg, 275 nmol/kg to 300 nmol/kg, 300 nmol/kg to 325 nmol/kg, 325 nmol/kg to 350 nmol/kg, 350 nmol/kg to 375 nmol/kg, 375 nmol/kg to 400 nmol/kg, 400 nmol/kg to 425 nmol/kg, 425 nmol/kg to 450 nmol/kg, 450 nmol/kg to 475 nmol/kg, 475 nmol/kg to 500 nmol/kg, 500 nmol/kg to 525 nmol/kg, 525 nmol/kg to 550 nmol/kg, 550 nmol/kg to 575 nmol/kg, 575 nmol/kg to 600 nmol/kg, 600 nmol/kg to 625 nmol/kg, 625 nmol/kg to 650 nmol/kg, 650 nmol/kg to 675 nmol/kg, 675 nmol/kg to 700 nmol/kg, 700 nmol/kg to 725 nmol/kg, 725 nmol/kg to 750 nmol/kg, 750 nmol/kg to 775 nmol/kg, 775 nmol/kg to 800 nmol/kg, 800 nmol/kg to 825 nmol/kg, 825 nmol/kg to 850 nmol/kg, 850 nmol/kg to 875 nmol/kg, 875 nmol/kg to 900 nmol/kg, 900 nmol/kg to 925 nmol/kg, 925 nmol/kg to 950 nmol/kg, 950 nmol/kg to 975 nmol/kg, 975 nmol/kg to 1000 nmol/kg, 1000 nmol/kg to 1025 nmol/kg, 1025 nmol/kg to 1050 nmol/kg, 1050 nmol/kg to 1075 nmol/kg, 1075 nmol/kg to 1100 nmol/kg, 1100 nmol/kg to 1125 nmol/kg, 1125 nmol/kg to 1150 nmol/kg, 1150 nmol/kg to 1175 nmol/kg, 1175 nmol/kg to 1200 nmol/kg, 1200 nmol/kg to 1225 nmol/kg, 1225 nmol/kg to 1250 nmol/kg, 1250 nmol/kg to 1275 nmol/kg, 1275 nmol/kg to 1300 nmol/kg, 1300 nmol/kg to 1325 nmol/kg, 1325 nmol/kg to 1350 nmol/kg, 1350 nmol/kg to 1375 nmol/kg, 1375 nmol/kg to 1400 nmol/kg, 1400 nmol/kg to 1425 nmol/kg, 1425 nmol/kg to 1450 nmol/kg, 1450 nmol/kg to 1475 nmol/kg, 1475 nmol/kg to 1500 nmol/kg, 1500 nmol/kg to 1525 nmol/kg, 1525 nmol/kg to 1550 nmol/kg, 1550 nmol/kg to 1575 nmol/kg, 1575 nmol/kg to 1600 nmol/kg, 1600 nmol/kg to 1625 nmol/kg, 1625 nmol/kg to 1650 nmol/kg, 1650 nmol/kg to 1675 nmol/kg, 1675 nmol/kg to 1700 nmol/kg, 1700 nmol/kg to 1725 nmol/kg, 1725 nmol/kg to 1750 nmol/kg, 1750 nmol/kg to 1775 nmol/kg, 1775 nmol/kg to 1800 nmol/kg, 1800 nmol/kg to 1825 nmol/kg, 1825 nmol/kg to 1850 nmol/kg, 1850 nmol/kg to 1875 nmol/kg, 1875 nmol/kg to 1900 nmol/kg, 1900 nmol/kg to 1925 nmol/kg, and 1925 nmol/kg to 2000 nmol/kg.

In some embodiments, the insulin analog of the present invention comprises a concentration of about 1 IU/mg to 3 IU/mg, 3 IU/mg to 5 IU/mg, 5 IU/mg to 7 IU/mg, 7 IU/mg to 9 IU/mg, 9 IU/mg to 11 IU/mg, 11 IU/mg to 13 IU/mg, 13 IU/mg to 15 IU/mg, 15 IU/mg to 17 IU/mg, 17 IU/mg to 19 IU/mg, 19 IU/mg to 21 IU/mg, 21 IU/mg to 23 IU/mg, 23 IU/mg to 25 IU/mg, 25 IU/mg to 27 IU/mg, 27 IU/mg to 29 IU/mg, 29 IU/mg to 31 IU/mg, 31 IU/mg to 33 IU/mg, 33 IU/mg to 35 IU/mg, 35 IU/mg to 37 IU/mg, 37 IU/mg to 39 IU/mg, 39 IU/mg to 41 IU/mg, 41 IU/mg to 43 IU/mg, 43 IU/mg to 45 IU/mg, 45 IU/mg to 47 IU/mg, 47 IU/mg to 49 IU/mg, 49 IU/mg to 51 IU/mg, 51 IU/mg to 53 IU/mg, 53 IU/mg to 55 IU/mg, 55 IU/mg to 57 IU/mg, 57 IU/mg to 59 IU/mg, 59 IU/mg to 61 IU/mg, 61 IU/mg to 63 IU/mg, 63 IU/mg to 65 IU/mg, 65 IU/mg to 67 IU/mg, 67 IU/mg to 69 IU/mg, 69 IU/mg to 71 IU/mg, 71 IU/mg to 73 IU/mg, 73 IU/mg to 75 IU/mg, 75 IU/mg to 77 IU/mg, 77 IU/mg to 79 IU/mg, 79 IU/mg to 81 IU/mg, 81 IU/mg to 83 IU/mg, 83 IU/mg to 85 IU/mg, 85 IU/mg to 87 IU/mg, 87 IU/mg to 89 IU/mg, 89 IU/mg to 91 IU/mg, 91 IU/mg to 93 IU/mg, 93 IU/mg to 95 IU/mg, 95 IU/mg to 97 IU/mg, 97 IU/mg to 99 IU/mg, and 99 IU/mg to 100 IU/mg.

In some embodiments, the insulin analog of the present invention comprises a concentration that is the human effective dose.

### Pharmaceutical Compositions

The disclosure herein also describes pharmaceutical compositions comprising an insulin analog. The compositions of the invention are prepared by conventional processes, involving dissolving and mixing the ingredients to give the desired composition.

In typical embodiments, the pharmaceutical composition comprises an insulin analog and a pharmaceutically acceptable vehicle or carrier. In certain embodiments, the pharmaceutically acceptable carrier is a microsphere, a micelle, or a nanoparticle. In certain embodiments, the pharmaceutically acceptable carrier is a protein, such as a receptor or albumin.

In some embodiments, the pharmaceutical composition comprises any one of an isotonic agent, an excipient, a preservative, or a buffer. In some embodiments, the isotonic agent is one or more of propylene glycol, sodium chloride, potassium chloride, mannitol, sorbitol, trehalose, lactitol, xylitol, glycerol, sucrose, glycine, lactose, glucose, maltose, lysine, isoleucine, aspartic acid, L-glycine, L-histidine, arginine, myo-inositol, or polyethylene glycol. In some embodiments, the preservative comprises any one of phenol, meta-cresol, ra-cresol, thiomerosal, methyl paraben, propyl paraben, butyl paraben, chlorobutanol, and phenoxyethanol. In some embodiments, the buffer comprises any one of dipotassium phosphate, sodium bicarbonate, and disodium phosphate. In some embodiments, the pharmaceutical composition comprises zinc acetate.

In some embodiments, the pharmaceutical composition comprises a surfactant. In certain embodiments, the surfactant is at least one of polysorbate, poloxamers, ethylene/polypropylene block polymers, lecithins, alcohols, sodium lauryl sulfate, bile acids and salts thereof, polymeric surfactants, long-chain fatty acids, phospholipids, ethoxylated castor oil, polyglycolyzed glycerides, acetylated monoglycerides, sorbitan fatty acid esters, polyoxyethylene sorbitan fatty acid esters, monoglycerides, diglycerides, glycerol, glycerophospholipids, glyceroglycolipids, sphingophospholipids, sphingoglycolipids, docusate sodium, docusate calcium, docusate potassium.

In some embodiments, the pharmaceutical composition comprises a chelating agent. Suitable examples of chelating agents include ethylenediaminetetraacetic acid or its salts, and mixtures thereof.

In typical embodiments, the pharmaceutical composition remains stable after exposure to multiple free-thaw cycles. In typical embodiments, the pharmaceutical composition remains stable after exposure to an elevated temperature. Stability can be measured in any way, including visually inspecting the compositions in daylight with a dark background for any signs of turbidity, changes in color or clarity, or any other visible precipitates.

In certain embodiments, the pharmaceutical composition is at a pH range from about 7.0 to about 11.0. In certain embodiments, the pH is adjusted to about one of the following values: 7.0, 7.2, 7.3, 7.4, 7.5, 7.6. 7.7, 7.8, 7.9, 8.0.

In certain embodiments, the pharmaceutical composition pH is adjusted using a pH-adjusting agent selected from the group consisting of sodium hydroxide, potassium hydroxide, hydrochloric acid, and N-methyl glucamine.

In some embodiments, the pharmaceutical composition comprises an anti-diabetic agent or an anti-obesity agent.

### Methods of Use

The disclosure herein also provides for methods of treatment of human disease or disorder using a therapeutically effective amount of the insulin analog. In typical embodiments, the human disease or disorder is diabetes and/or obesity.

In typical embodiments, the method of treating diabetes comprises administering to a patient a therapeutically effective amount of an insulin analog.

In certain embodiments, the treatment results in a reduction of measured HbA1C in the patient. In certain embodiments, the patient's glucose levels are measured by a clinician before administration of the insulin analog.

In certain embodiments, the treatment results in any one of decreasing food intake, reduction in body weight, suppression of appetite, or inducing satiety in the patient.

In some embodiments, administration of the insulin analogs of the present invention provide a longer lasting glucose lowering effect compared to administration of insulin degludec. In some embodiments, the glucose lowering effect of the insulin analogs of the present invention lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 1 of the present invention provides a longer lasting glucose lowering effect compared to administration of insulin degludec. In some embodiments, the glucose lowering effect of insulin analog 1 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 2 of the present invention provides a longer lasting glucose lowering effect compared to administration of insulin degludec. In some embodiments, the glucose lowering effect of insulin analog 2 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 3 of the present invention provides a longer lasting glucose lowering effect compared to administration of insulin degludec. In some embodiments, the glucose lowering effect of insulin analog 3 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 4 of the present invention provides a longer lasting glucose lowering effect compared to administration of insulin degludec. In some embodiments, the glucose lowering effect of insulin analog 4 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days. In some embodiments, the glucose lowering effect of insulin analog 4 lasts 48 hours.

In some embodiments, administration of insulin analog APi3545 of the present invention provides a longer lasting glucose lowering effect compared to administration of insulin degludec. In some embodiments, the glucose lowering effect of insulin analog APi3545 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days. In some embodiments, the glucose lowering effect of insulin analog APi3545 lasts 48 hours.

In some embodiments, administration of insulin analog APi3546 of the present invention provides a longer lasting glucose lowering effect compared to administration of insulin degludec. In some embodiments, the glucose lowering effect of insulin analog APi3546 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog APi3547 of the present invention provides a longer lasting glucose lowering effect compared to administration of insulin degludec. In some embodiments, the glucose lowering effect of insulin analog APi3547 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog APi3549 of the present invention provides a longer lasting glucose lowering effect compared to administration of insulin degludec. In some embodiments, the glucose lowering effect of insulin analog APi3549 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

Methods of detecting glucose levels are known in the art and comprise blood glucose meters, test strips, ELISA assays, etc.

In some embodiments, administration of the insulin analogs of the present invention provide a longer lasting c-peptide lowering effect compared to administration of insulin degludec. In some embodiments, the c-peptide lowering effect of the insulin analogs of the present invention lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 1 of the present invention provides a longer lasting c-peptide lowering effect compared to administration of insulin degludec. In some embodiments, the c-peptide lowering effect of insulin analog 1 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 2 of the present invention provides a longer lasting c-peptide lowering effect compared to administration of insulin degludec. In some embodiments, the c-peptide lowering effect of insulin analog 2 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 3 of the present invention provides a longer lasting c-peptide lowering effect compared to administration of insulin degludec. In some embodiments, the c-peptide lowering effect of insulin analog 3 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog 4 of the present invention provides a longer lasting c-peptide lowering effect compared to administration of insulin degludec. In some embodiments, the c-peptide lowering effect of insulin analog 4 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog APi3545 of the present invention provides a longer lasting c-peptide lowering effect compared to administration of insulin degludec. In some embodiments, the c-peptide lowering effect of insulin analog APi3545 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days. In some embodiments, the c-peptide lowering effect of insulin analog APi3545 lasts 72 hours.

In some embodiments, administration of insulin analog APi3546 of the present invention provides a longer lasting c-peptide lowering effect compared to administration of insulin degludec. In some embodiments, the c-peptide lowering effect of insulin analog APi3546 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog APi3547 of the present invention provides a longer lasting c-peptide lowering effect compared to administration of insulin degludec. In some embodiments, the c-peptide lowering effect of insulin analog APi3547 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

In some embodiments, administration of insulin analog APi3549 of the present invention provides a longer lasting c-peptide lowering effect compared to administration of insulin degludec. In some embodiments, the c-peptide lowering effect of insulin analog APi3549 lasts 6 hours, 12 hours, 24 hours, 48 hours, 72 hours, 96 hours, 5 days, 6 days, 7 days, 8 days, 9 days, 10 days, 11 days, 12 days, 13 days, or 14 days.

Methods of detecting c-peptide levels are known in the art and comprise test strips, ELISA assays, etc.

In some embodiments, administration of the insulin analogs of the present invention provide a lower body weight increase compared to administration of insulin degludec. In some embodiments, the lower body weight increase of the insulin analogs of the present invention lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog 1 of the present invention provides a lower body weight increase compared to administration of insulin degludec. In some embodiments, the lower body weight increase of insulin analog 1 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog 2 of the present invention provides a lower body weight increase compared to administration of insulin degludec. In some embodiments, the lower body weight increase of insulin analog 2 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog 3 of the present invention provides a lower body weight increase compared to administration of insulin degludec. In some embodiments, the lower body weight increase of insulin analog 3 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog 4 of the present invention provides a lower body weight increase compared to administration of insulin degludec. In some embodiments, the lower body weight increase of insulin analog 4 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog APi3545 of the present invention provides a lower body weight increase compared to administration of insulin degludec. In some embodiments, the lower body weight increase of insulin analog APi3545 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog APi3546 of the present invention provides a lower body weight increase compared to administration of insulin degludec. In some embodiments, the lower body weight increase of insulin analog APi3546 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog APi3547 of the present invention provides a lower body weight increase compared to administration of insulin degludec. In some embodiments, the lower body weight increase of insulin analog APi3547 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, administration of insulin analog APi3549 of the present invention provides a lower body weight increase compared to administration of insulin degludec. In some embodiments, the lower body weight increase of insulin analog APi3549 lasts at least 6 hours, at least 12 hours, at least 24 hours, at least 48 hours, at least 72 hours, at least 96 hours, at least 5 days, at least 6 days, at least 7 days, at least 8 days, at least 9 days, at least 10 days, at least 11 days, at least 12 days, at least 13 days, or at least 14 days.

In some embodiments, the insulin analogs of the present invention provide no insulin-like growth factor-1 receptor (IGF1R) activation. In some embodiments insulin analog 1 provides to IGF1R activation. In some embodiments, insulin analog 2 provides no IGF1R activation. In some embodiments, insulin analog 3 provides no IGF1R activation. In some embodiments, insulin analog 4 provide no IGF1R activation.

In some embodiments, the insulin analog is administered by oral, intravenous, subcutaneous, parenteral, transdermal, intramuscular, rectal, vaginal, or topical administration. In some embodiments, the insulin analog is delivered by a transdermal patch. In some embodiments, the insulin analog is delivered by an implant device or method.

In some embodiments, the insulin analog is administered by injection. In some embodiments, the insulin analog is administered by injection by a syringe. In some embodiments, the insulin analog is administered orally by tablet, pill, capsule, elixir, syrup, extract, or solution. In some embodiments, the insulin analog is administered by injection at an interval of once daily, twice daily, or once a week, or twice a week, or three times a week, or four times a week, or five times a week. In certain embodiments, the insulin analog is administered by injection once a week. In some embodiments, the insulin analog is administered by injection once every two weeks or once every three weeks. In some embodiments, the insulin analog is administered by injection once every month.

In some embodiments, the insulin analog is administered by injection. In some embodiments, the insulin analog is administered by injection by a syringe. In some embodiments, the insulin analog is administered orally by tablet, pill, capsule, elixir, syrup, extract, or solution. In some embodiments, the insulin analog is administered by injection at an interval of once every 12 hours. In some embodiments, the insulin analog is administered by injection at an interval of once every 24 hours. In some embodiments, the insulin analog is administered by injection at an interval of once every 48 hours. In some embodiments, the insulin analog is administered by injection at an interval of once every 72 hours. In some embodiments, the insulin analog is administered by injection at an interval of once every 96 hours. In some embodiments, the insulin analog is administered by injection at an interval of once every 5 days. In some embodiments, the insulin analog is administered by injection at an interval of once every 6 days. In some embodiments, the insulin analog is administered by injection at an interval of once every 7 days. In some embodiments, the insulin analog is administered by injection at an interval of once every 8 days. In some embodiments, the insulin analog is administered by injection at an interval of once every 9 days. In some embodiments, the insulin analog is administered by injection at an interval of once every 10 days. In some embodiments, the insulin analog is administered by injection at an interval of once every 11 days. In some embodiments, the insulin analog is administered by injection at an interval of once every 12 days. In some embodiments, the insulin analog is administered by injection at an interval of once every 13 days. In some embodiments, the insulin analog is administered by injection at an interval of once every 14 days. In some embodiments, the insulin analog is administered by injection at an interval of once every 15 days. In some embodiments, the insulin analog is administered by injection at an interval of once every 16 days. In some embodiments, the insulin analog is administered by injection at an interval of once every 17 days. In some embodiments, the insulin analog is administered by injection at an interval of once every 18 days. In some embodiments, the insulin analog is administered by injection at an interval of once every 19 days. In some embodiments, the insulin analog is administered by injection at an interval of once every 20 days. In some embodiments, the insulin analog is administered by injection at an interval of once every 21 days. In some embodiments, the insulin analog is administered by injection at an interval of once every 22 days. In some embodiments, the insulin analog is administered by injection at an interval of once every 23 days. In some embodiments, the insulin analog is administered by injection at an interval of once every 24 days. In some embodiments, the insulin analog is administered by injection at an interval of once every 25 days. In some embodiments, the insulin analog is administered by injection at an interval of once every 26 days. In some embodiments, the insulin analog is administered by injection at an interval of once every 27 days. In some embodiments, the insulin analog is administered by injection at an interval of once every 28 days. In some embodiments, the insulin analog is administered by injection at an interval of once every 29 days. In some embodiments, the insulin analog is administered by injection at an interval of once every 30 days. In some embodiments, the insulin analog is administered by injection at an interval of twice weekly. In some embodiments, the insulin analog is administered by injection at an interval of once weekly. In some embodiments, the insulin analog is administered by injection at an interval of twice monthly. In some embodiments, the insulin analog is administered by injection at an interval of once monthly. In some embodiments, the insulin analog is administered by injection at an interval of once every other month. In some embodiments, the insulin analog is administered by injection at an interval of twice yearly. In some embodiments, the insulin analog is administered by injection at an interval of once yearly.

In some embodiments, the compositions, formulations, or pharmaceutical compositions disclosed herein are useful in the field of medicine for the treatment, prophylaxis, palliation, or amelioration of metabolic diseases and/or disorders.

The present disclosure provides a method for administering to a subject in need thereof, including a human subject, the compositions, formulations, or pharmaceutical compositions disclosed herein to slow, stop, or reverse disease progression. As a nonlimiting example, disease progression can be measured by tests or diagnostic tool(s) known to those skilled in the art. As another non-limiting example, disease progression can be measured by changes in the pathological features of the pancreas, brain, or other tissues of the subject.

In some embodiments, a therapeutically effective amount of the insulin analog is administered to a subject. In some embodiments, the effective amount to be employed therapeutically will depend, for example, upon the therapeutic context and objectives. One skilled in the art will appreciate that the appropriate dosage levels for treatment, according to some embodiments, will thus vary depending, in part, upon the molecule delivered, the indication for which the insulin analog is being used, the route of administration, and the size (body weight, body surface or organ size) and/or condition (the age and general health) of the subject. In some embodiments, the clinician can titer the dosage and modify the route of administration to obtain the optimal therapeutic effect.

In some embodiments, a therapeutically effective amount of compositions, formulations, or pharmaceutical compositions disclosed herein inhibits and/or prevents a particular disorder, and/or the symptoms of the disorder.

In some embodiments, the insulin analog and the compositions, formulations, or pharmaceutical compositions disclosed herein comprising the insulin analogs are delivered systemically. For example, in some embodiments, the insulin analogs are administered through intravenous injection. In some embodiments, the insulin analogs are administered through subcutaneous injection. In some embodiments, insulin analogs are administered through intramuscular injection. In some embodiments, the insulin analogs are administered through intravenous infusion. In some embodiments, the systemically delivered insulin analogs are capable of crossing the blood-brain barrier.

### Kits and Methods of Manufacture

In some embodiments, methods of making the insulin analogs and the compositions, formulations, or pharmaceutical compositions disclosed herein are envisioned.

The insulin analog and the compositions, formulations, or pharmaceutical compositions disclosed herein can be designed and/or synthesized using any suitable method known in the art.

In some embodiments, the disclosure provides a kit comprising a insulin analog and a composition, formulation, or pharmaceutical composition disclosed herein comprising the insulin analog. In some embodiments, a kit includes a insulin analog and a composition, formulation, or pharmaceutical composition disclosed herein comprising the insulin analog, and instructions for use. In some embodiments, the kits comprise, in a suitable container, a insulin analog and a composition, formulation, or pharmaceutical composition disclosed herein comprising the insulin analog, one or more controls, and various buffers, reagents, enzymes and other standard ingredients known in the art.

The container can include at least one vial, well, test tube, flask, bottle, syringe, or other container means, into which a insulin analog and a composition, formulation, or pharmaceutical composition disclosed herein comprising the insulin analog can be placed, and in some instances, suitably aliquoted. Where an additional component is provided, the kit can contain additional containers into which this component can be placed. The kits can also include a means for containing a insulin analog and any other reagent containers in close confinement for commercial sale. Such containers can include injection or blow-molded plastic containers into which the desired vials are retained. Containers and/or kits can include labeling with instructions for use and/or warnings.

In some embodiments, a kit comprises a container comprising a insulin analog and a pharmaceutically acceptable carrier, or a pharmaceutical composition comprising the insulin analog, and instructions for treating or delaying progression of a metabolic disease in a subject in need thereof. In some embodiments, a kit comprises a container comprising a insulin analog and a pharmaceutically acceptable carrier, or a pharmaceutical composition comprising the insulin analog, and instructions for administering the insulin analog to a subject in need thereof, alone or in combination with another agent, for treating or delaying progression of a metabolic disease in the subject.

### Definitions

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “about,” unless indicated otherwise, refers to the recited value, e.g., amount, dose, temperature, time, percentage, etc., ±10%, ±9%, ±8%, ±7%, ±6%, ±5%, 4%, ±3%, ±2%, or ±1%.

As used herein, the term “subject” herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats and horses. Preferred agricultural animals would be pigs, cattle and goats. A “patient” is a human subject.

The phrases “therapeutically effective amount” and “effective amount” and the like, as used herein, indicate an amount necessary to administer to a patient, or to a cell, tissue, or organ of a patient, to achieve a therapeutic effect, such as an ameliorating or alternatively a curative effect. The effective amount is sufficient to elicit the biological or medical response of a cell, tissue, system, animal, or human that is being sought by a researcher, veterinarian, medical doctor, or clinician. Determination of the appropriate effective amount or therapeutically effective amount is within the routine level of skill in the art.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

### Exemplary Embodiments

Exemplary Embodiment No. 1. An analog of human insulin comprising a peptide A-chain and a peptide B-chain, wherein the A-chain from N-terminus to C-terminus comprises
A1-A2-A3-A4-A5-A6-A7-A8-A9-A10-A11-A12-A13-A14-A15-A16-A17-A18-A19-A20-A21;
wherein the B-chain from N-terminus to C-terminus comprises
B1-B2-B3-B4-B5-B6-B7-B8-B9-B10-B11-B12-B13-B14-B15-B16-B17-B18-B19-B20-B21-B22-B23-B24-B25-B26-B27-B28-B29;
wherein
A1 is Gly,
A2 is Ile,
A3 is Val,
A4 is Glu,
A5 is Gln,
A6 is Cys,
A7 is Cys,
A8 is Thr,
A9 is Ser,
A10 is Ile,
A11 is Cys,
A12 is Ser,
A13 is Leu,
A14 is Gla, or Aad, or Ppa
A15 is Gln,
A16 is Leu,
A17 is Glu,
A18 is Asn,
A19 is Tyr,
A20 is Cys,
A21 is Asn; and
wherein
B1 is Phe,
B2 is Val,
B3 is Asn,
B4 is Gln,
B5 is His,
B6 is Leu,
B7 is Cys,
B8 is Gly,
B9 is Ser,
B10 is His,
B11 is Leu,
B12 is Val,
B13 is Glu,
B14 is Ala,
B15 is Leu,
B16 is His(3-Me), or Pal, or His(5-I),
B17 is Leu,
B18 is Val,
B19 is Cys,
B20 is Gly,
B21 is Glu,
B22 is Arg,
B23 is Gly,
B24 is Phe,
B25 is His(3-Me), or Pal, or 2Fal, or Thi, or Ala(4-thiazoyl), or 3-Pal,
B26 is Tyr,
B27 is Thr,
B28 is Pro, or Hyp, or Pip,
B29 is Lys.

Exemplary Embodiment No. 2. The insulin analog of the previous Exemplary Embodiment, wherein the B-chain N-terminus or the B-chain C-terminus or both the N-terminus and C-terminus is substituted with a substituent.

Exemplary Embodiment No. 3. The insulin analog of any one of the previous Exeplary Embodiments, wherein the substituent is a AEEA-AEEA-γ-Glu-(CH2)ₘ—OH wherein m is 12, 14, 16, 18, 20, or 22.

Exemplary Embodiment No. 4. The insulin analog of any one of the previous Exemplary Embodiments, wherein A6 and A1 l are connected by a linker.

Exemplary Embodiment No. 5. The insulin analog of any one of the previous Exemplary Embodiments, wherein A7 and B7 are connected by a linker.

Exemplary Embodiment No. 6. The insulin analog of any one of the previous Exemplary Embodiments, wherein A20 and B19 are connected by a linker.

Exemplary Embodiment No. 7. The insulin analog of any one of the previous Exemplary Embodiments, wherein the linker is cleavable or non-cleavable.

Exemplary Embodiment No. 8. The insulin analog of any one of the previous Exemplary Embodiments, wherein the linker comprises a disulfide bond.

Exemplary Embodiment No. 9. The insulin analog of any one of the previous Exemplary Embodiments, wherein the insulin analog binding affinity (EC₅₀) to insulin receptor is below 10 nM.

Exemplary Embodiment No. 10. The insulin analog of any one of the previous Exemplary Embodiments, wherein the insulin analog binding affinity (IC₅₀) to insulin receptor is below 5 nM.

Exemplary Embodiment No. 11. The insulin analog of any one of the previous Exemplary Embodiments, wherein the insulin analog binding affinity (IC₅₀) to insulin receptor is below 1 nM.

Exemplary Embodiment No. 12. The insulin analog of any one of the previous Exemplary Embodiments, wherein the insulin analog binding affinity is measured by surface plasmon resonance.

Exemplary Embodiment No. 13. The insulin analog of any one of the previous Exemplary Embodiments, wherein the insulin analog binding affinity is measured using a recombinant cell line.

Exemplary Embodiment No. 14. The insulin analog of the previous Exemplary Embodiments, wherein the recombinant cell line expresses insulin receptor.

Exemplary Embodiment No. 15. The insulin analog of the previous Exemplary Embodiments, wherein the insulin analog binding affinity (IC₅₀) to the insulin receptor is determined by measuring luciferase activity.

Exemplary Embodiment No. 16. The insulin analog of any one of the previous Exemplary Embodiments, wherein the insulin analog exhibits an in vivo plasma elimination half-life of at least 1 hour, or 2 hours, or 4 hours, or 6 hours, or 8 hours, or 10 hours, or 24 hours, or 48 hours, or 72 hours, or 168 hours in human.

Exemplary Embodiment No. 17. A pharmaceutical composition comprising an insulin analog of any one of the previous Exemplary Embodiments and a pharmaceutically acceptable vehicle or carrier.

Exemplary Embodiment No. 18. A pharmaceutical composition of the previous Exemplary Embodiments, further comprising any one of an isotonic agent, an excipient, a preservative, and a buffer.

Exemplary Embodiment No. 19. A pharmaceutical composition of any one of the previous Exemplary Embodiments, further comprising a surfactant.

Exemplary Embodiment No. 20. A pharmaceutical composition of any one of the previous Exemplary Embodiments, further comprising an anti-diabetic agent.

Exemplary Embodiment No. 21. A pharmaceutical composition of any one of the previous Exemplary Embodiments, wherein the pharmaceutically acceptable carrier is a microsphere, micelle, or nanoparticle.

Exemplary Embodiment No. 22. A method of treating diabetes, comprising administering to a patient a therapeutically effective amount of an insulin analog of any one of the previous Exemplary Embodiments.

Exemplary Embodiment No. 23. The method of the previous Exemplary Embodiments, wherein the treatment results in the reduction or stabilization of average blood glucose levels in the patient.

Exemplary Embodiment No. 24. The method of any one of the previous Exemplary Embodiments, wherein the insulin analog is administered by oral, subcutaneous, intravenous, parenteral, transdermal, intramuscular, rectal, vaginal, pump-based, or topical administration.

Exemplary Embodiment No. 25. The method of any one of the previous Exemplary Embodiments, wherein the insulin analog is administered by subcutaneous, transdermal, intramuscular, or intravenous injection.

Exemplary Embodiment No. 26. The method of any one of the previous Exemplary Embodiments, wherein the insulin analog is administered once weekly.

Exemplary Embodiment No. 27. The method of any one of the previous Exemplary Embodiments, wherein administration of the insulin analog provides a longer lasting glucose lowering effect compared to administration of degludec.

Exemplary Embodiment No. 28. The method of any one of the previous Exemplary Embodiments, wherein the administration of the insulin analog provides a longer lasting c-peptide lowering effect compared to administration of degludec.

Exemplary Embodiment No. 29. The method of any one of the previous Exemplary Embodiments, wherein the administration of the insulin analog provides a lower body weight increase compared to administration of degludec.

Exemplary Embodiment No. 30. The method of any one of the previous Exemplary Embodiments, wherein the administration of the insulin analog does not activate the insulin-like growth factor-1 receptor (IGF1R).

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or see, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art

### Example 1: Synthesis of Insulin Analogs

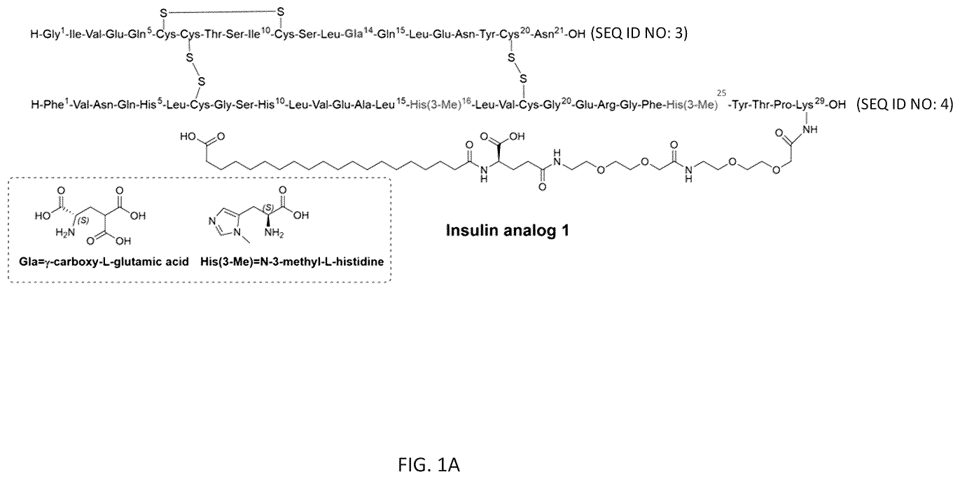

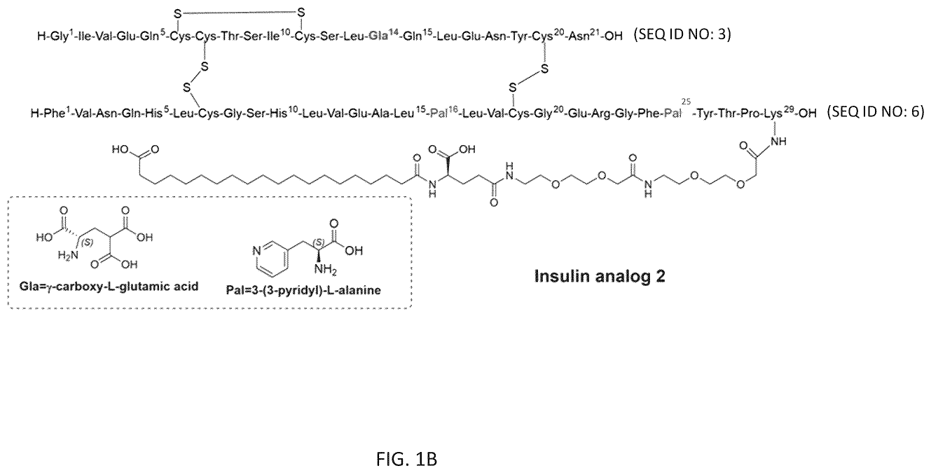

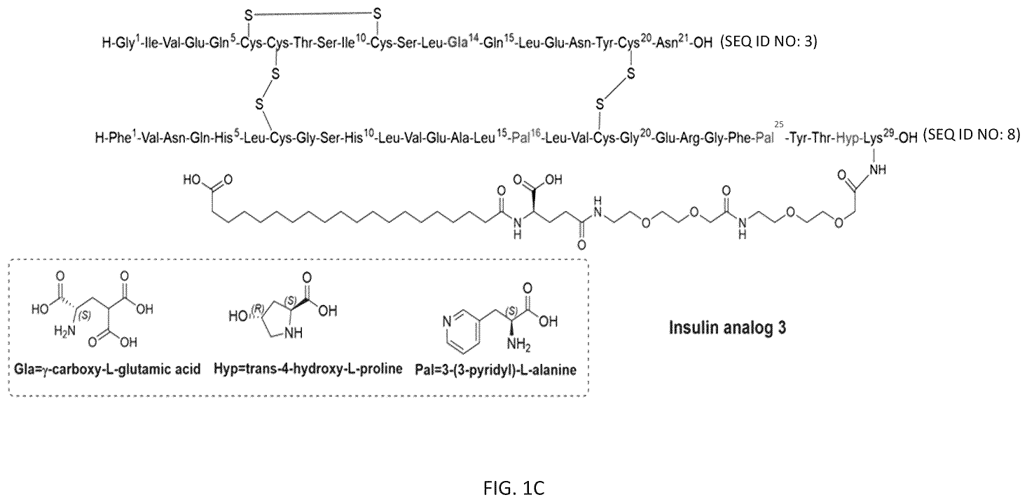

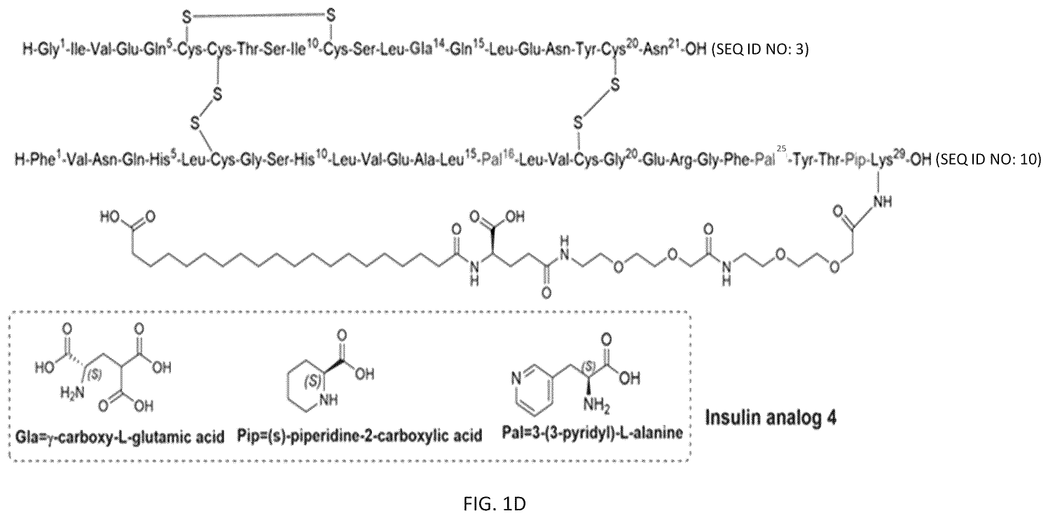

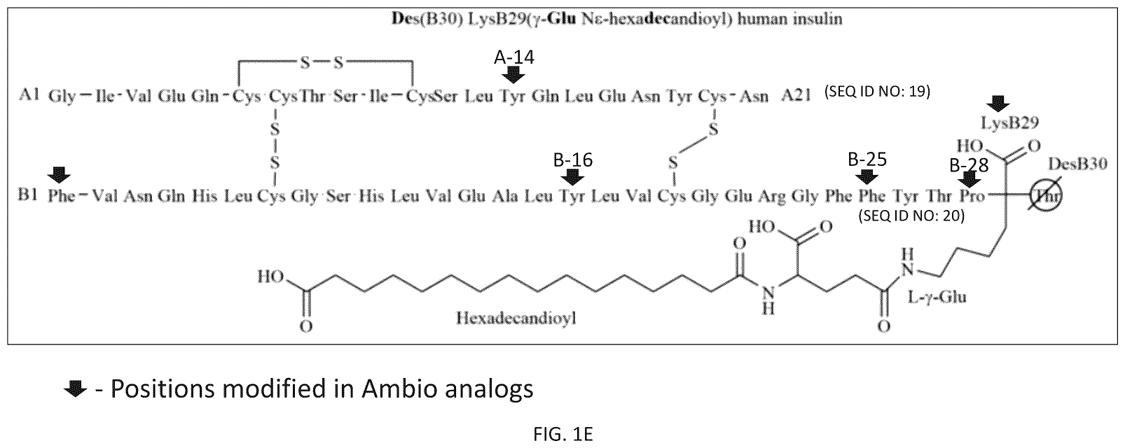

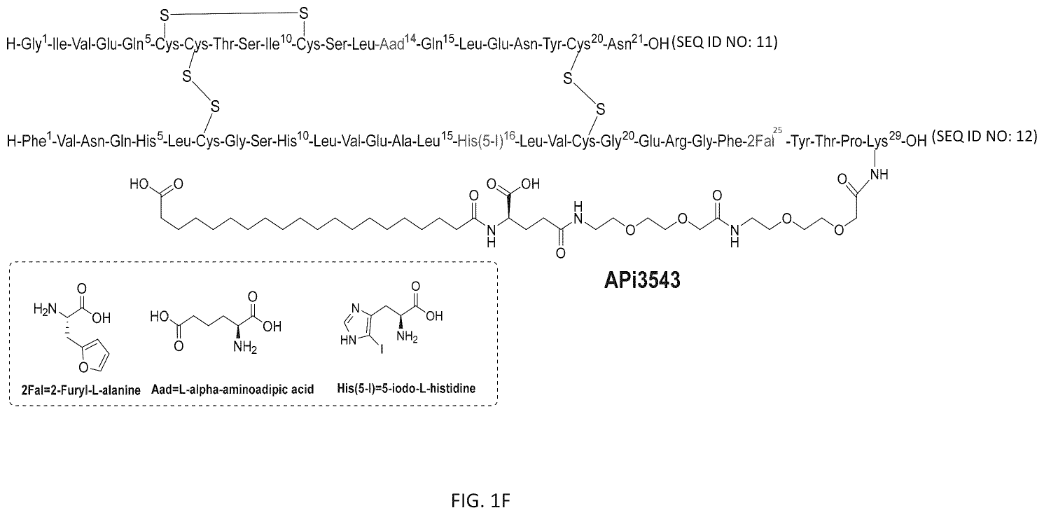

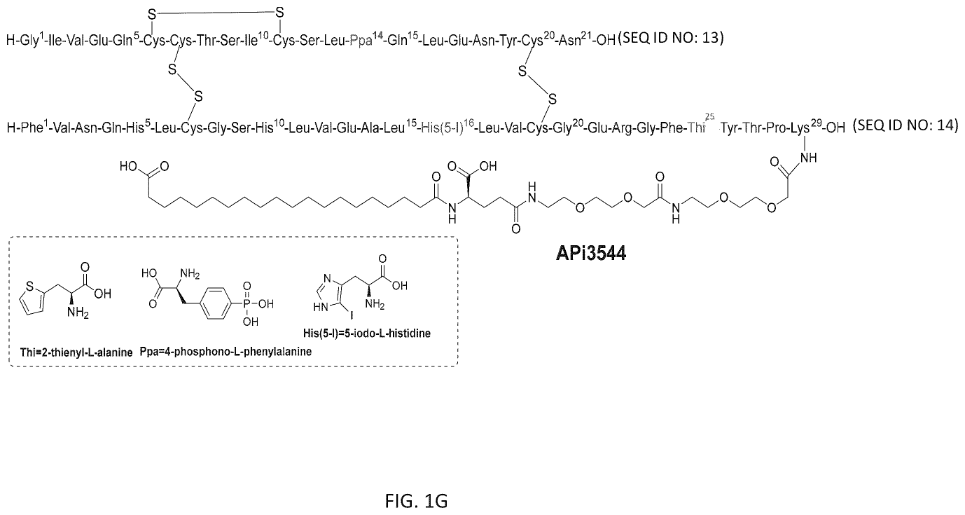

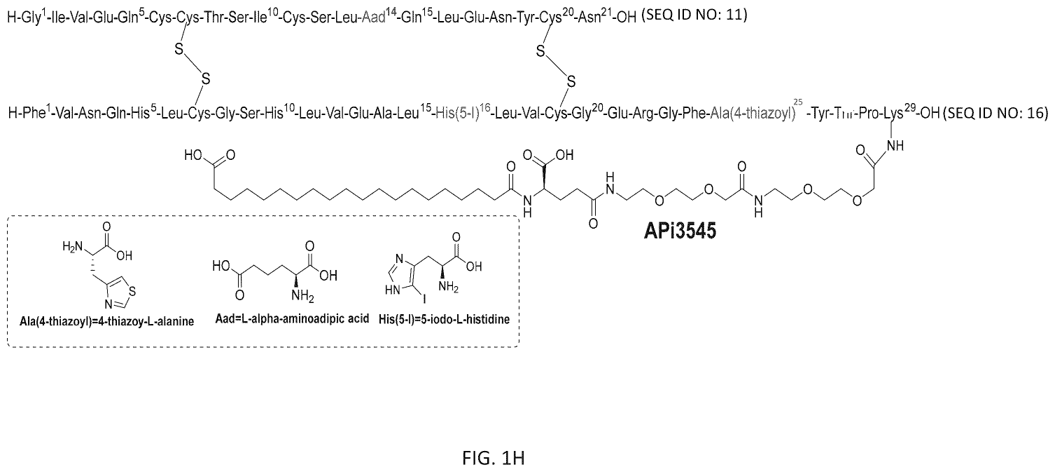

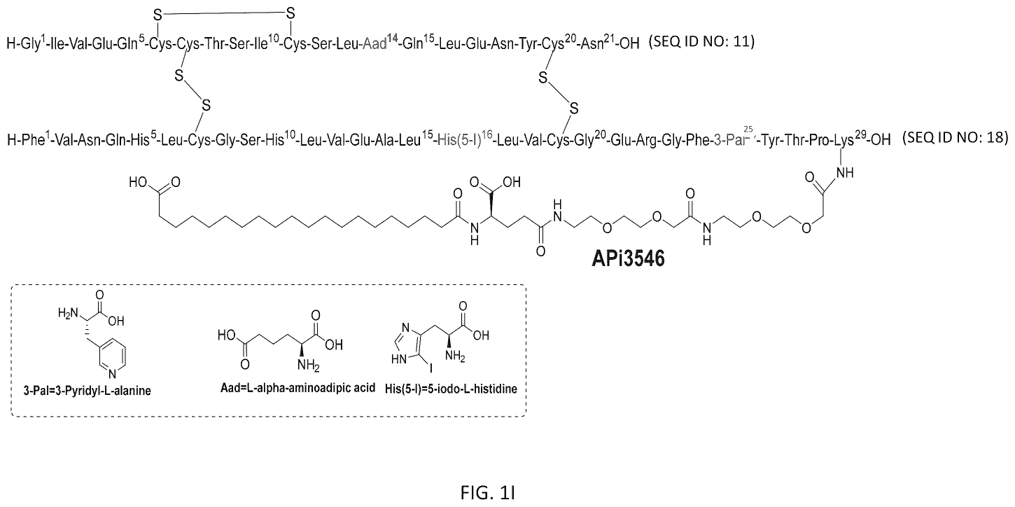

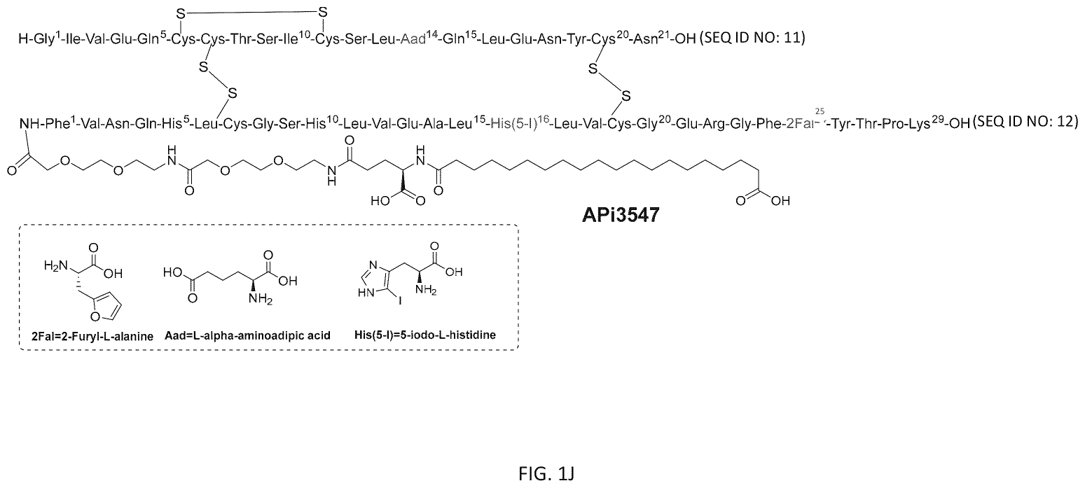

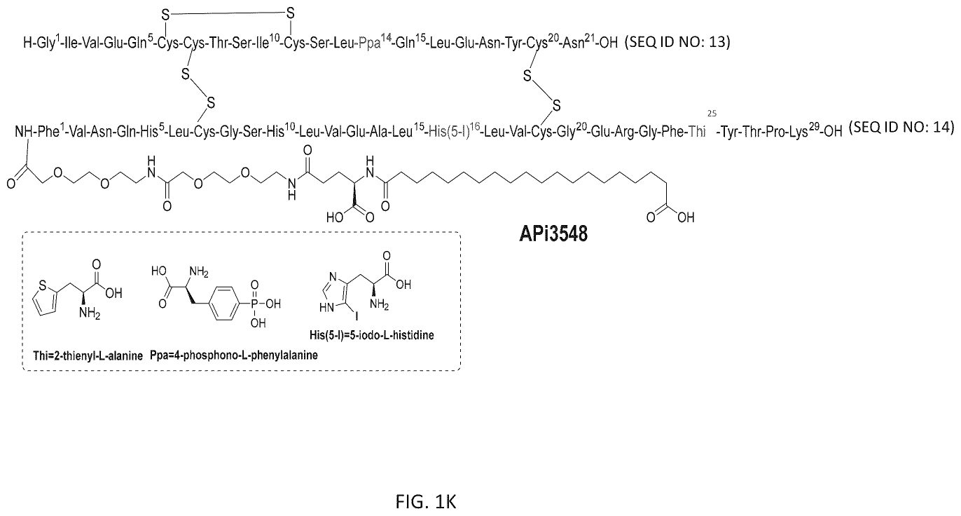

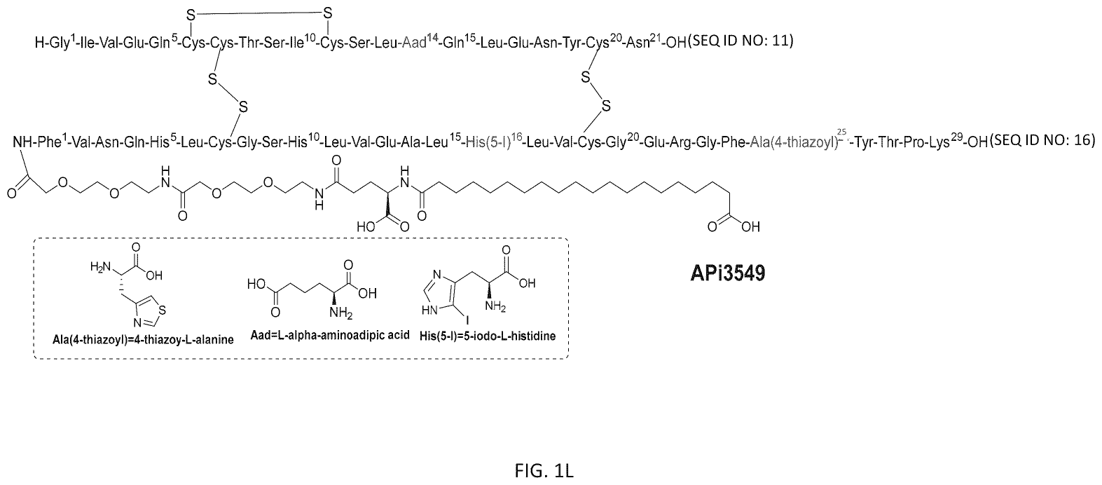

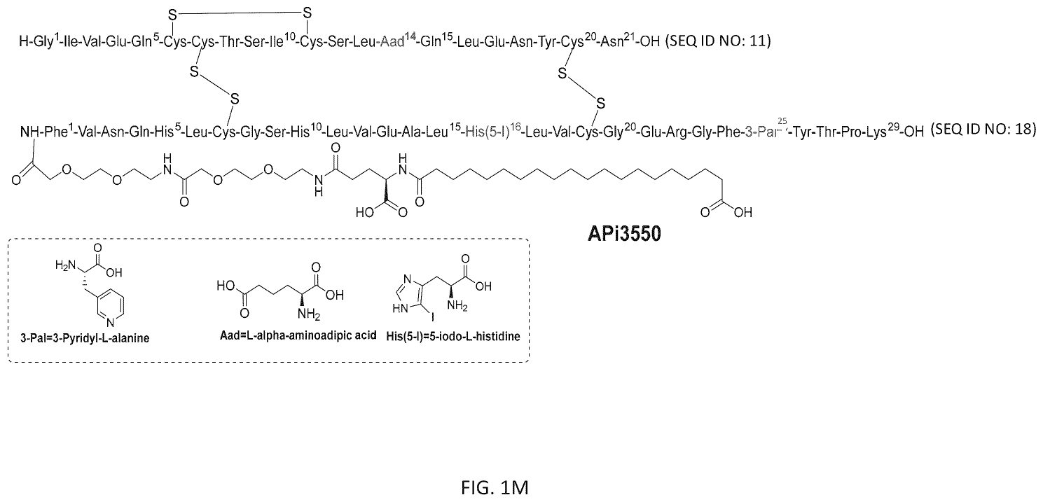

Insulin analogs as shown in FIGS. 1A-IM were synthesized by a combination of solid-phase synthesis and solution-phase synthesis. The purification of the analogs was performed by IPLC purification with certain buffers and the use of certain reverse-phase columns including the combination of ion-exchange and reverse-phase media packed into the column.

### Example 2: Biopotency of Eight Insulin Analogs

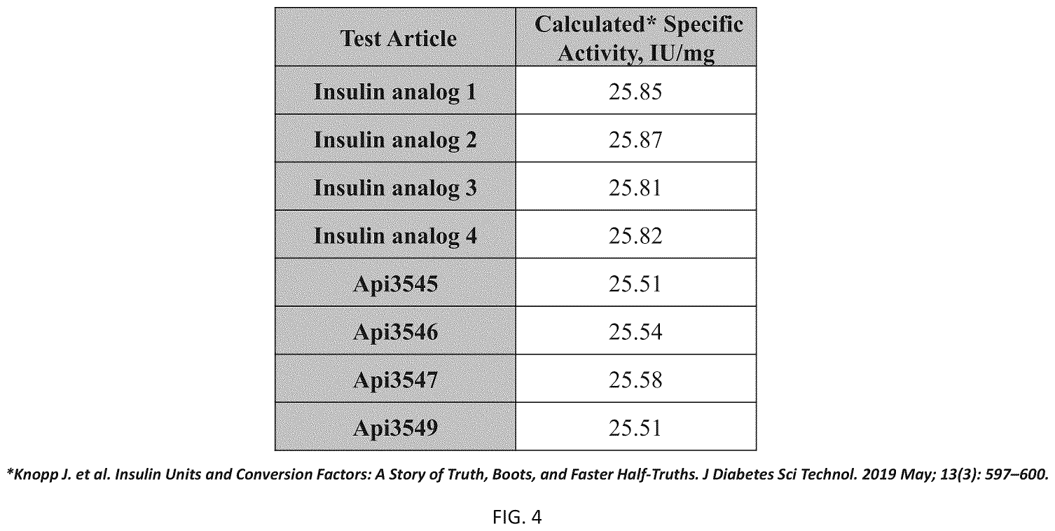

The biopotency of the eight insulin analogs as shown in FIGS. 1A-1D, FIGS. 1H-1J, and FIG. 1L, respectively, was determined. The eight insulin analogs were assigned a calculated insulin concentration unit (IU/mL), using the conversion factor of 1 μIU/L=6 picomole/L (J Diabetes Sci Technol. 2019 May; 13(3): 597-600, the contents of which are incorporated by reference in their entireties). FIG. 4 illustrates the calculated IU/mg for the eight insulin analogs.

The biopotency of the eight insulin analogs was determined by comparison with the insulin reference standard following chapter <1211> of Chinese Pharmacopeia. In particular, the insulin analogs and insulin reference standard were prepared with either diluent 1 or diluent 2. Diluent 1 comprises 0.9% sodium chloride at a pH of 2.5 (adjusted with HCl) and 0.2% phenol. Diluent 2 comprises 0.9% sodium chloride at a pH of 2.5 (adjusted with HCl). The insulin reference standard (27.2 IU/mg) and the insulin analogs (calculated as 25 IU/mg) were prepared with diluent 1. In addition, low- and high-dosing solutions (0.05 IU/mL and 0.1 IU/mL, respectively) of insulin reference standard and test analogs were prepared with diluent 2.

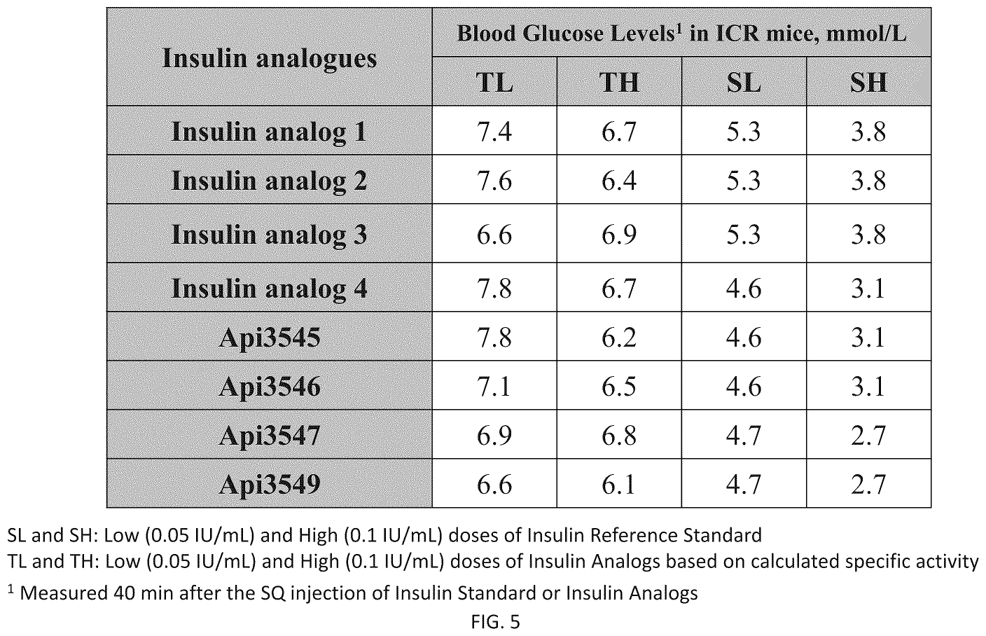

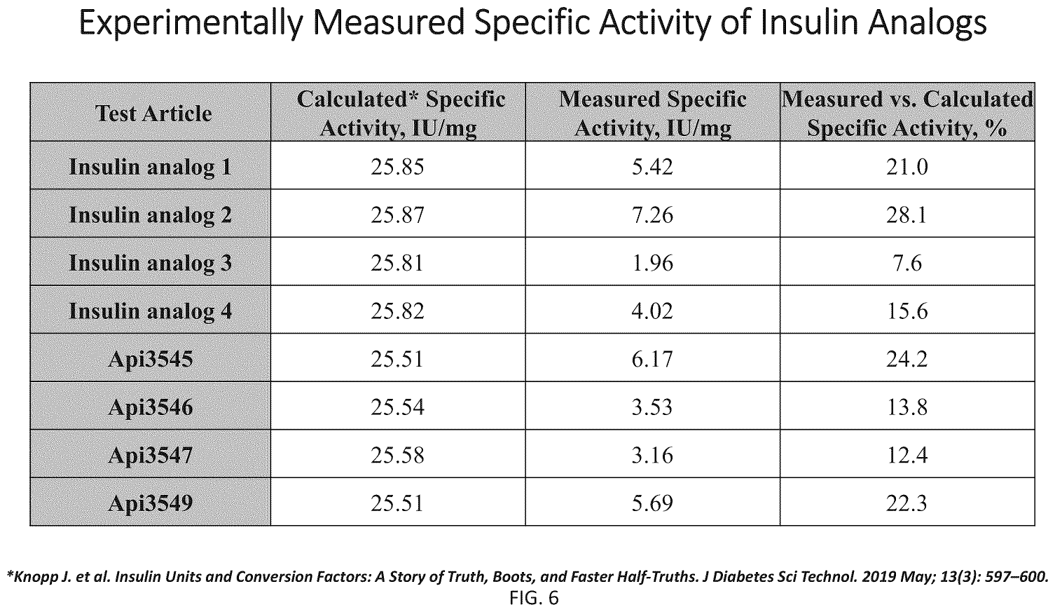

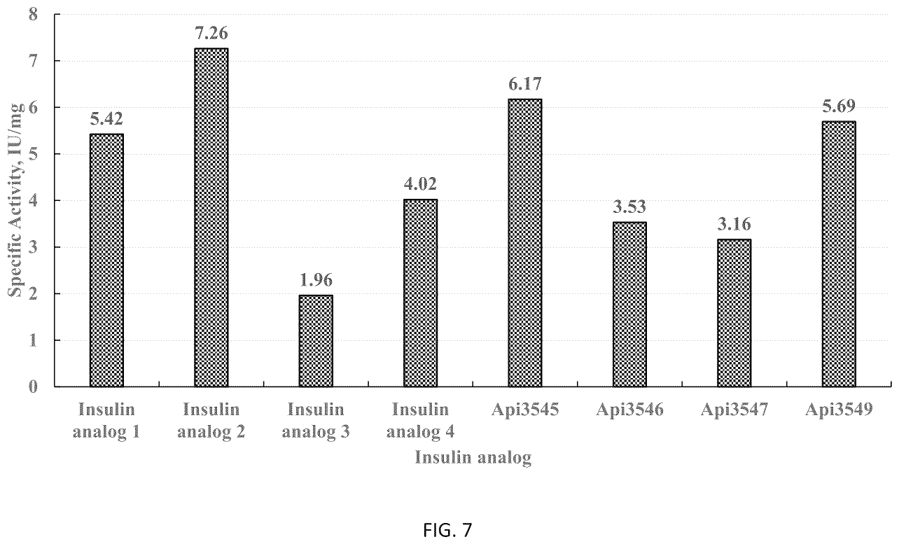

Following chapter <1211> of Chinese Pharmacopeia, ICR mice (25-28 g, 6 animals per group) were injected with 0.2-0.3 mL of low and high dosing solution of the eight insulin analogs and insulin standard. Low and high doses of insulin standard and insulin analogs were administered to the mice following parallel design/protocol. After administration, sample blood was collected from the orbital venous sinus forty minutes after each administration. The glucose concentration of each blood sample was determined by the glucose oxidase-peroxidase method (Roche blood glucose test strips). FIG. 5 illustrates the glucose levels in the blood following administration (i.e., after 40 minutes), as it relates to both high and low dose solutions for the analogs and reference standard, respectively. FIG. 6 summarizes the relative biopotency of the insulin analogs by comparing the calculated biopotency and measured biopotency. FIG. 7 demonstrates all eight tested insulin analogs have a glucose lowering effect with three of the most active insulin analogs being insulin analog 2 (FIG. 1), Api3545 (FIG. 1H), Api3549 (FIG. 1L).

### Example 3: Subcutaneous Administration of an Insulin Analog in a db/db Murine Model Demonstrates Controlled Glucose Profile Over Time

Murine db/db murine strain is a well-accepted type II diabetes model that recapitulates most aspects of human type II diabetes. In particular, db/db mice carry mutations in the leptin receptor that lead to obesity, decreased insulin receptor sensitivity, and subsequent increased levels of blood glucose, decreased β-cell function, and elevated HBA1c levels. Like human Type II diabetes, the diabetic condition in db/db mice is progressive. Animals have near normal or slightly elevated plasma glucose (PG) and relatively normal β-cell function at 6 weeks of age. Fasting plasma glucose (FPG) levels gradually elevate over several weeks and β-cell function declines such that by about 16 weeks of age FPG is very high (>400 mg/dL) and j-cell function has entirely degenerated. The db/db mouse model may be used in an acute setting but is more typically used in a chronic setting to evaluate a test compound's cumulative action over several weeks of treatment. The variability is reasonably low such that good statistically significant effects are seen with validating compounds with group sizes of about 8 animals.

Accordingly, the ability of insulin analog 1 (FIG. 1A) to control the glucose profile in a db/db type II diabetes model was tested. As a control, wild type m/m mouse strain was included in the study. Mice were male, between ages 6-8 weeks, and the total group size was 8. The insulin analogs were delivered as a subcutaneous single dose. The dose volume was 5 mL/Kg (125 L per 25 g mouse). Mice were injected at the following dose levels: Degludec 1.5 IU/kg (model) and insulin analog #1 (FIG. 1A) at 0.04 mg/kg, 0.12 mg/kg, or 0.36 mg/kg. All insulin compounds (Degludec and analog #1) were diluted with 0.9% sodium chloride (vehicle, normal saline). Vehicle controls were comprised of 0.9% sodium chloride.

Blood collection for glucose plasma concentration measurements occurred at Oh (0-60 min before dosing), 0.5 h, 1 h, 2 h, 4 h, 6 h, 8 h, 10 h, 12 h, 18 h, 24 h, 30 h after the single dose. Moreover, the mouse body weight was measured daily for 3 days.

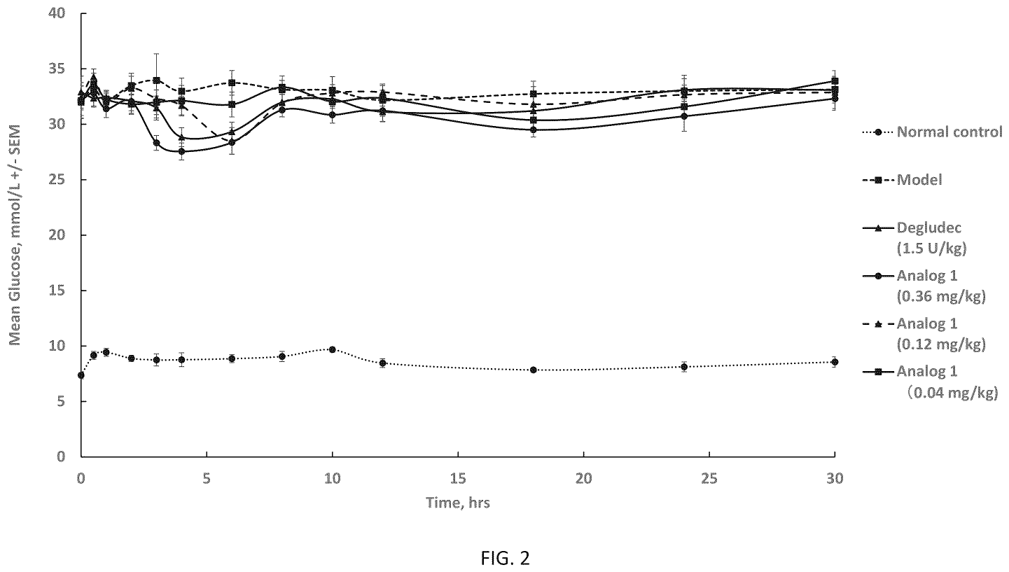

As shown in FIG. 2, the 2, 4, 6, and 8 hr time points glucose profiles for all 3 study groups, 1.5 IU/mg Degludec, 0.36 mg/kg, and 0.12 mg/kg Analog 1, are statistically significantly lower compared to db/db vehicle control. The 0.36 mg/kg Analog #1 glucose profile was statistically lower than db/db vehicle control during the entire time course starting from 2 hr time point. Moreover, the response (glucose level decrease) to 0.36 mg/kg, 0.12 mg/kg, and 0.04 mg/kg doses of Analog #1 were dose proportional.

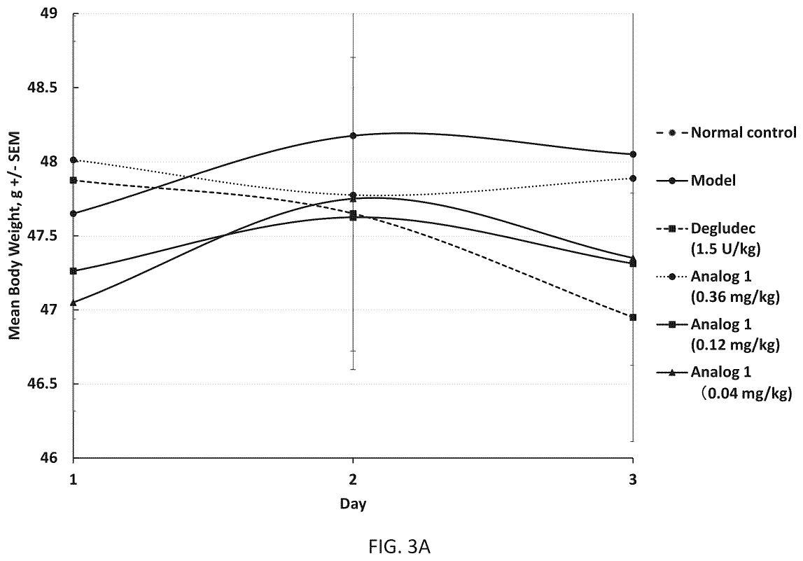

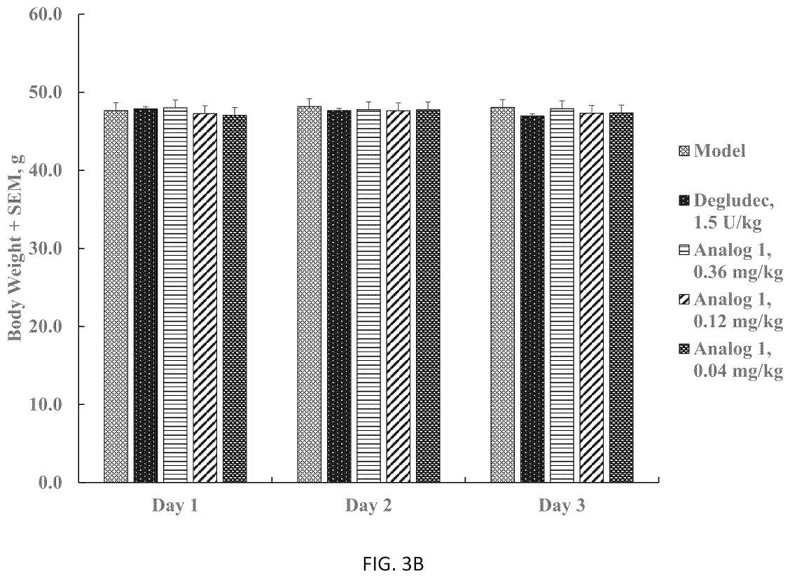

At least within the Day 1-3 time frame the treatment with 1.5 IU/mg Degludec and all dose levels of Analog #1 had no significant effect on animal body weight (FIGS. 3A-3B). The results demonstrate insulin analog #1 is capable of effective glucose profile regulation while maintaining safety (via body weight).

### Example 4: Insulin Analog Efficacy in Non-Fasted Sprague-Dawley Rats

Male Sprague-Dawley (SD) rats at the age of 8-10 weeks old were administered a dose of 175 nmol-200 nmol insulin analogs 1, 2, 3, 4, APi3545, APi3546, APi3547, and APi3549 through subcutaneous injection. Insulin Degludec was used as a positive control. A vehicle control consisted of glycerol (19.6 mg), metacresol (1.72 mg), phenol (1.50 mg), zinc acetate (91.75 pg), and WFI to 1 mL.

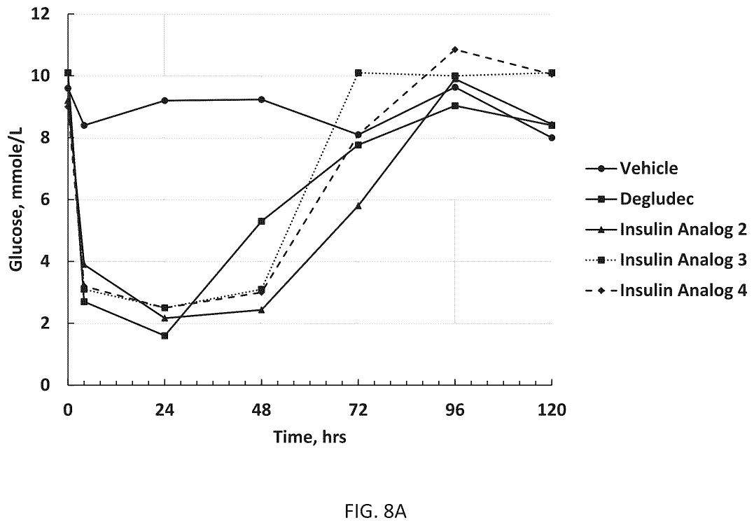

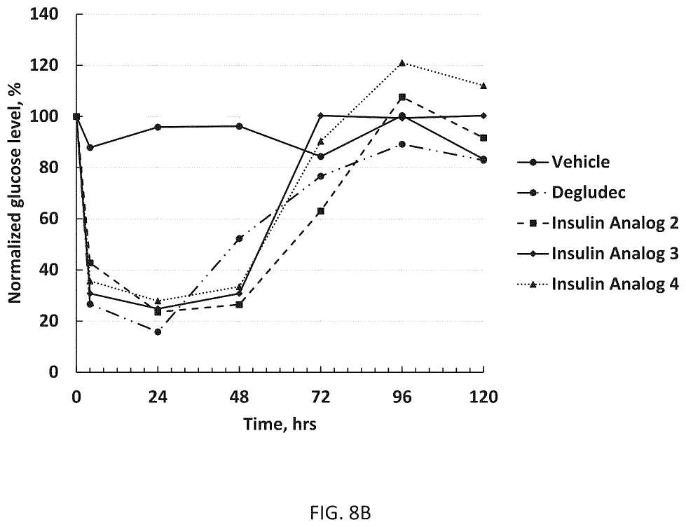

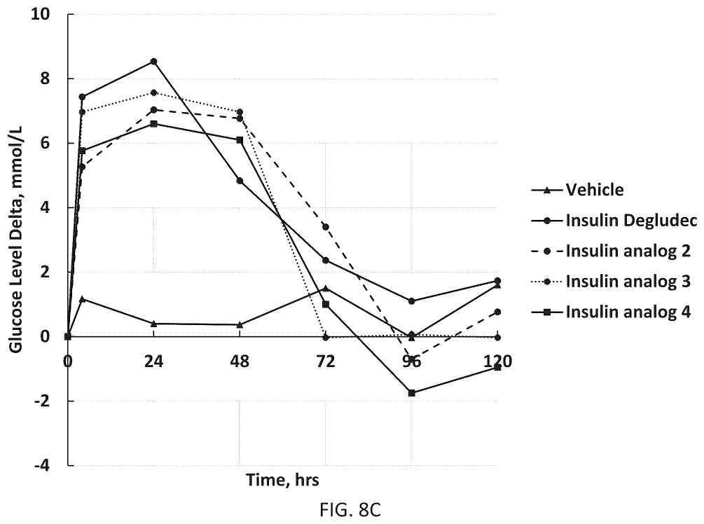

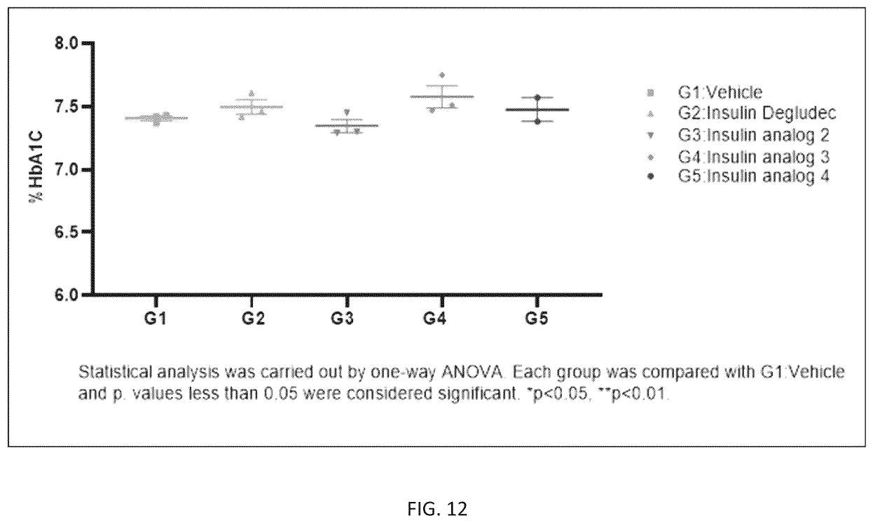

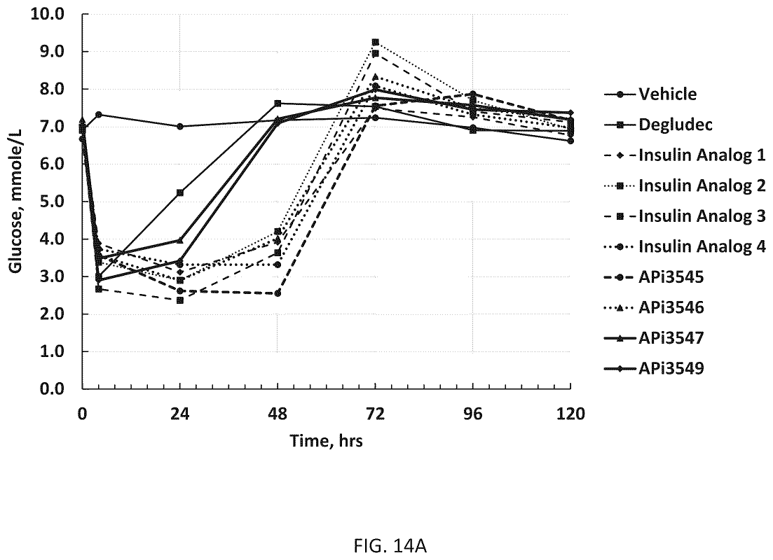

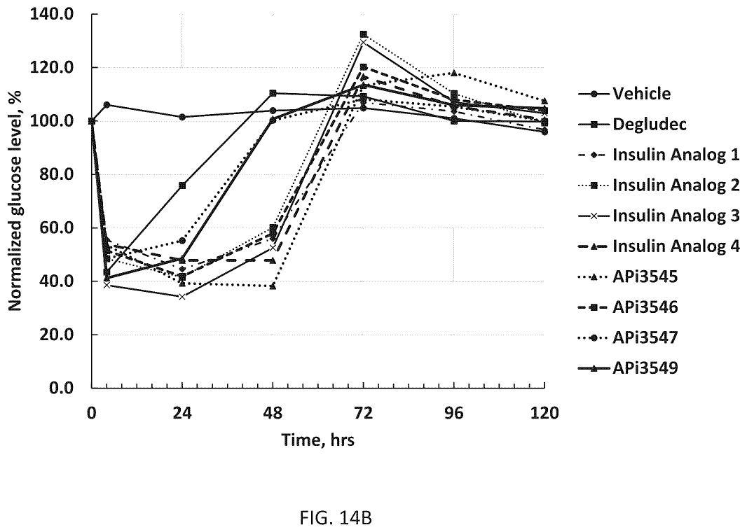

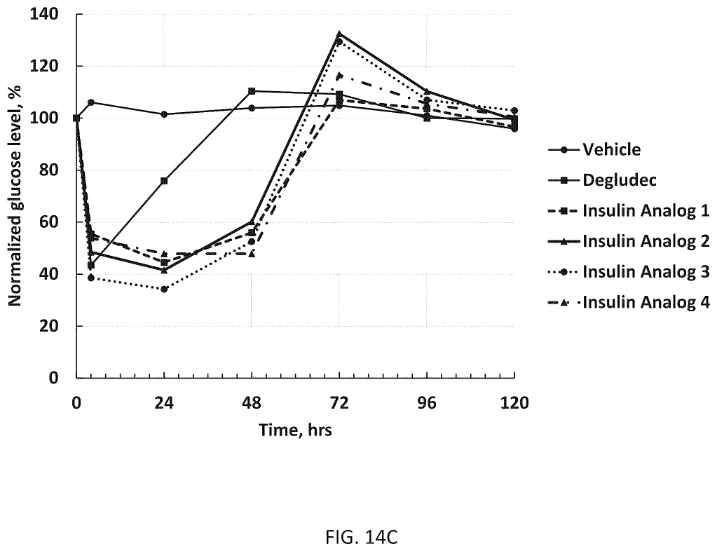

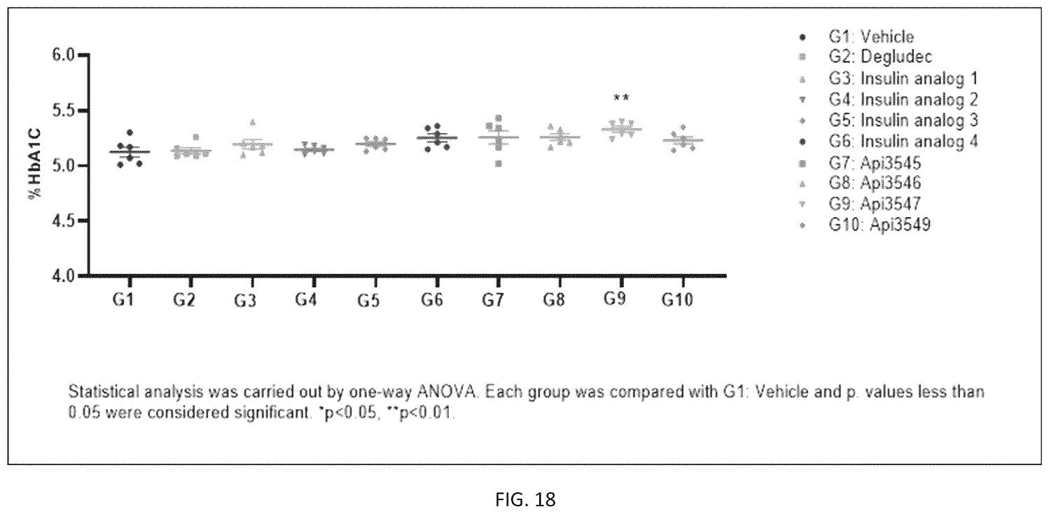

As shown in FIGS. 8A-8C, 12, 14A-14C, and 18, rats injected with insulin analogs 1, 2, 3, 4, APi3545, APi3546, APi3547, and APi3549 showed a longer lasting decrease in glucose levels compared to rats injected with Degludec. Insulin analog 4 and APi3545 maintained the low glucose level for up to 48 hours. Insulin analog 3 demonstrated the most profound glucose lowering effect.

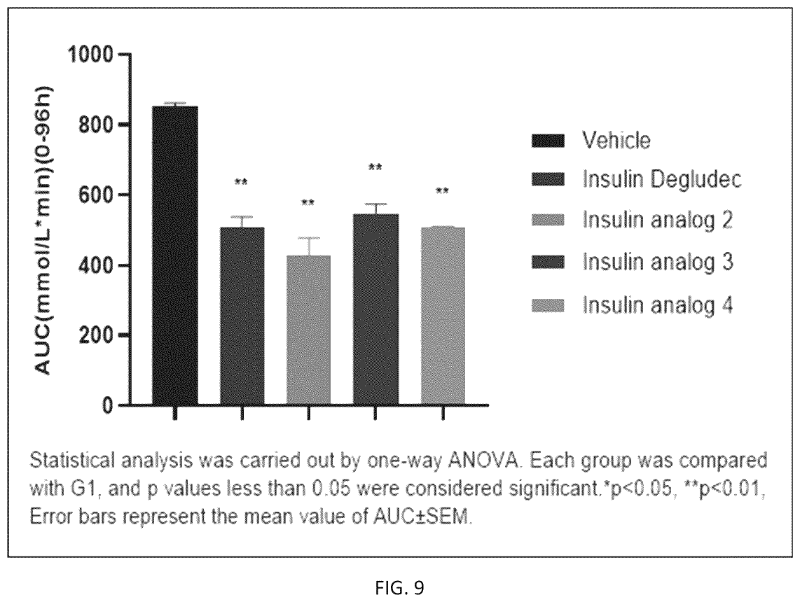

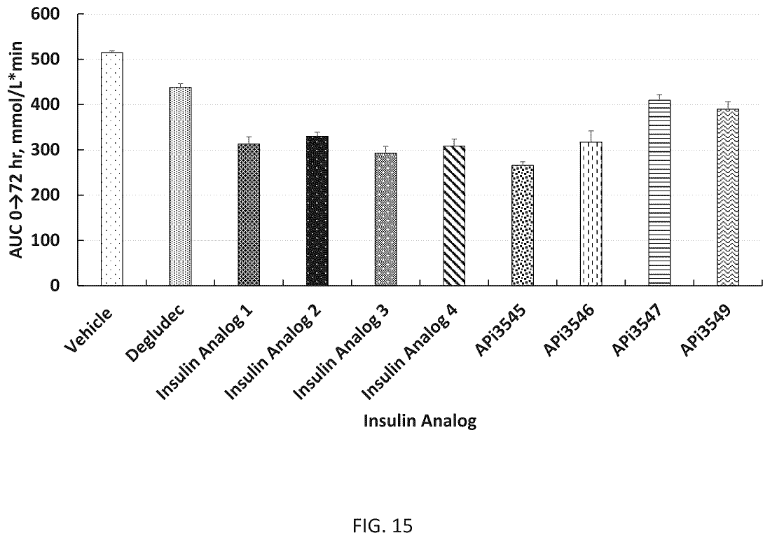

As shown in FIG. 9 and FIG. 15, glucose area under the curve (AUC) values of insulin analogs 2, 3, and 4 follow the order of their specific activities. Additionally, rats injected with insulin analogs 1, 2, 3, 4, APi3545, APi3546, APi3547, and APi3549 showed a longer lasting decrease in glucose levels compared to rats injected with Degludec. Insulin analog 4 and APi3545 maintained the low glucose level for up to 48 hours. Insulin analog 3 demonstrated the most profound glucose lowering effect.

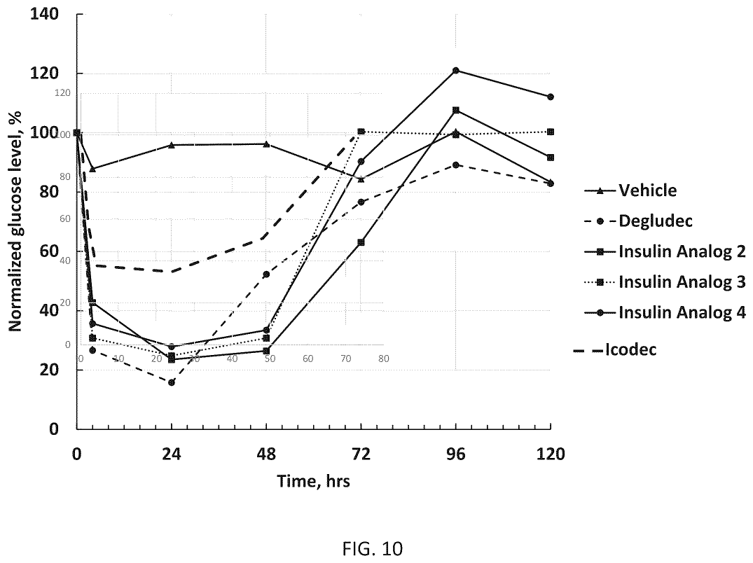

As shown in FIG. 10, rats injected with insulin analog 2 showed a longer lasting decrease in glucose level compared to rats injected with Degludec.

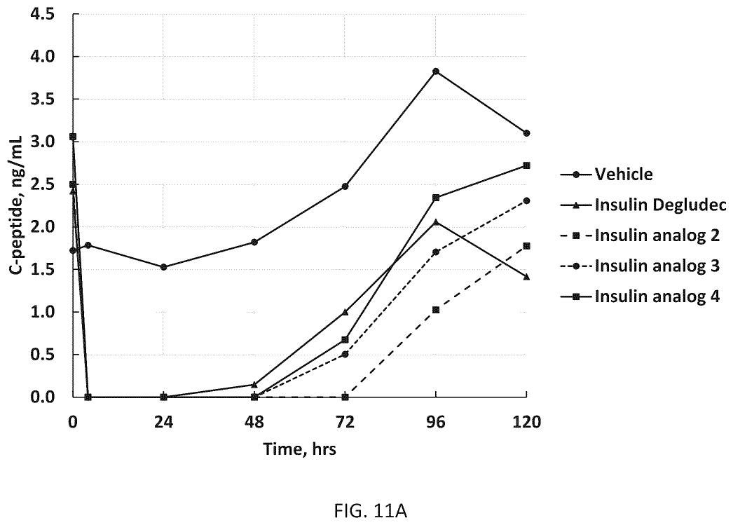

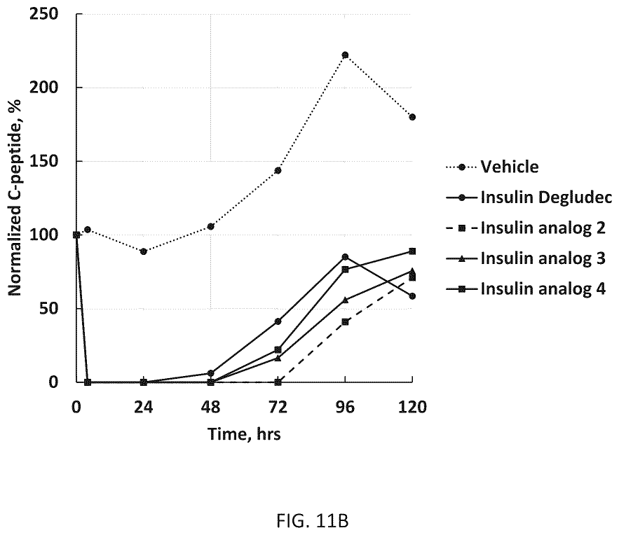

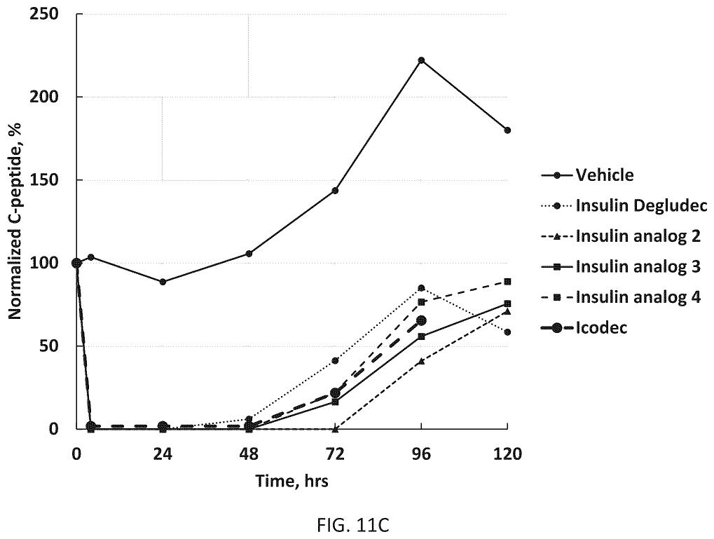

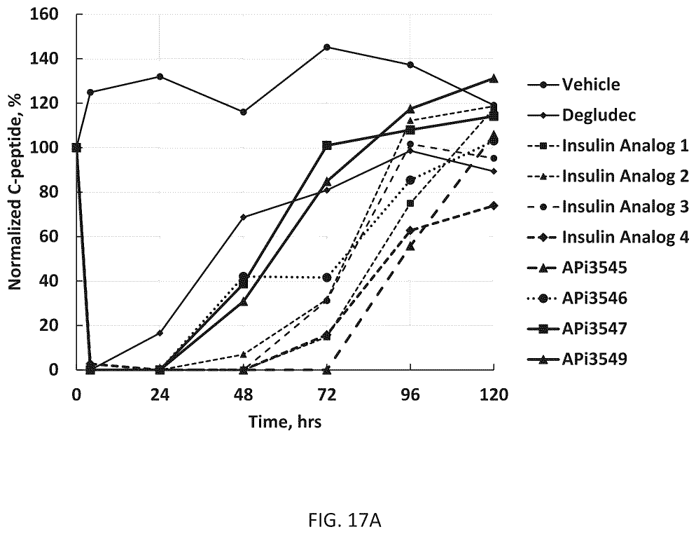

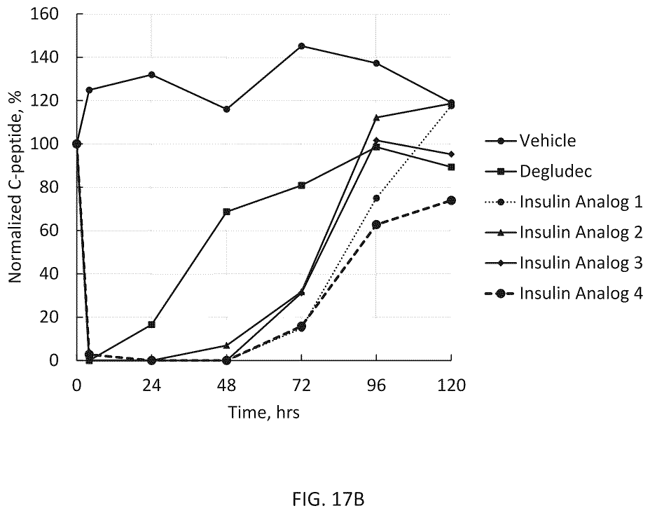

As shown in FIGS. 11A-11C and FIGS. 17A-17B, rats injected with insulin analogs 1, 2, 3, 4, APi3545, APi3546, APi3547, and APi3549 maintained the level of c-peptide for a longer time compared to rats injected with Degludec. Insulin analog 2 showed the most profound and longest-lasting c-peptide lowering effect. Insulin analog APi3545 maintained an undetectable c-peptide level for up to 72 hours.

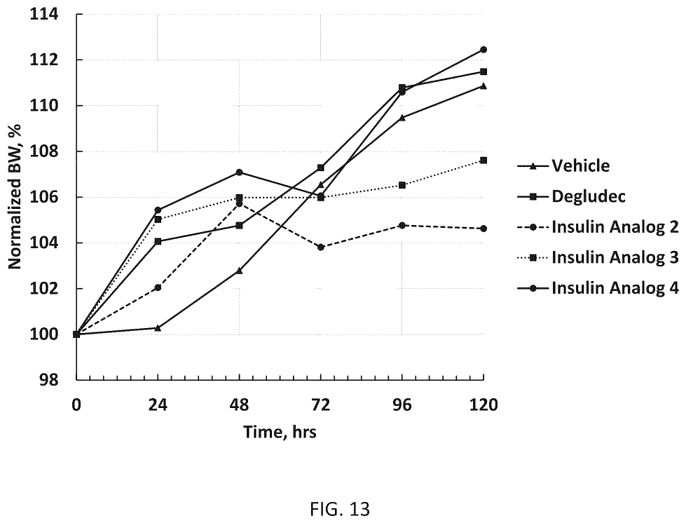

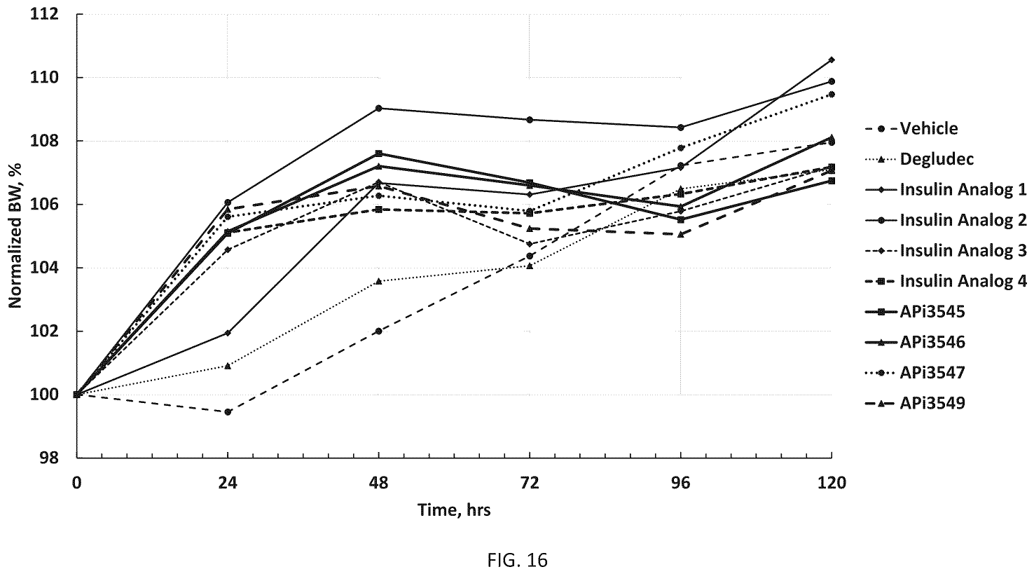

As shown in FIG. 13 and FIG. 16, rats injected with insulin analogs 1, 2, 4, APi3547, and APi3546 show a lower rate of body weight increase compared to rats injected with Degludec.

### Example 5: Structure-Activity Relationship

The attachment of a fatty acid to the N-terminus of the B chain (APi3547 and APi3549) substantially eliminates glucose-lowering activity compared to the analogs with the fatty acid attached to B-Lys²⁹.

Pip to Hyp replacement in position B-28 enhances the glucose lowering effect of insulin analogs with the fatty acid attached to B-Lys²⁹.

Replacement of Gly to Aad in A-14, His(3-Me) to Pal to His (5-I) in B-16, and His(3-Me) to Pal to 3-Pal in B-25 are almost equivalent in the insulin analogs with the fatty acid residue attached to B-Lys²⁹.

Replacements in Degludec (Tyr→Aad in A14, Tyr→His (5-I) in B-16, and Phe→Ala (4-thiazoyl) in B-25 resulted in the most long-acting insulin analog tested (APi3545).

Compared to Degludec, all structural variations tested enhanced and prolonged the glucose-lowering effect in insulin analogs.

### Example 6: In Vitro Insulin Analog Interaction with Igf-1R Receptor

Insulin-like growth factor (IGF-1R) is a tyrosine kinase type receptor that regulates cell growth and proliferation and can be activated by IGF-1, IGF-2, and insulin.

The IGF-1R signaling pathway regulates numerous cellular phenotypes associated with tumor cell survival and growth—including cell cycle progression, apoptosis, and differentiation.

Epidemiological studies have reported a positive association between circulating IGF-1 levels and various primary cancers, such as breast, colorectal, and prostate cancer.

IGFR-1R receptor activation by insulin analogs, prospective T1D and T2D drugs, could be considered by the Agency as a potential carcinogenesis risk.

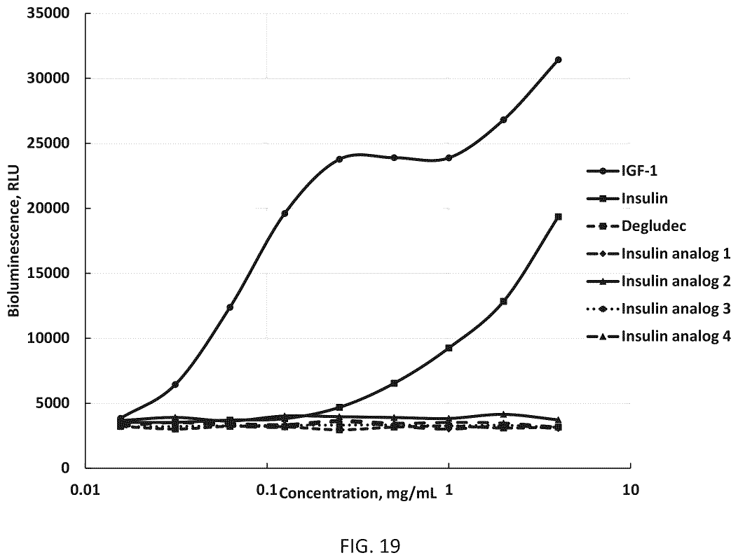

IGF-1R (Luc) HEK293 reporter cells were exposed to 0.015-4 mg/mL IGF-1, insulin, and insulin analogs 1, 2, 3, and 4. As shown in FIG. 19, none of insulin analogs 1, 2, 3, and 4 showed IGF-1R activation up to 4 μg/mL (1.6 μM) concentration.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. An analog of human insulin comprising a peptide A-chain and a peptide B-chain, wherein the A-chain from N-terminus to C-terminus comprises
A1-A2-A3-A4-A5-A6-A7-A8-A9-A10-A11-A12-A13-A14-A15-A16-A17-A18-A19-A20-A21;
wherein the B-chain from N-terminus to C-terminus comprises
B1-B2-B3-B4-B5-B6-B7-B8-B9-B10-B11-B12-B13-B14-B15-B16-B17-B18-B19-B20-B21-B22-B23-B24-B25-B26-B27-B28-B29;
wherein
A1 is Gly,
A2 is Ile,
A3 is Val,
A4 is Glu,
A5 is Gln,
A6 is Cys,
A7 is Cys,
A8 is Thr,
A9 is Ser,
A10 is Ile,
A11 is Cys,
A12 is Ser,
A13 is Leu,
A14 is Gla, or Aad, or Ppa,
A15 is Gln,
A16 is Leu,
A17 is Glu,
A18 is Asn,
A19 is Tyr,
A20 is Cys,
A21 is Asn; and
wherein
B1 is Phe,
B2 is Val,
B3 is Asn,
B4 is Gln,
B5 is His,
B6 is Leu,
B7 is Cys,
B8 is Gly,
B9 is Ser,
B10 is His,
B11 is Leu,
B12 is Val,
B13 is Glu,
B14 is Ala,
B15 is Leu,
B16 is His(3-Me), or Pal, or His(5-I),
B17 is Leu,
B18 is Val,
B19 is Cys,
B20 is Gly,
B21 is Glu,
B22 is Arg,
B23 is Gly,
B24 is Phe,
B25 is His(3-Me), or Pal, or 2Fal, or Thi, or Ala(4-thiazoyl), or 3-Pal,
B26 is Tyr,
B27 is Thr,
B28 is Pro, or Hyp, or Pip,
B29 is Lys.

2. The insulin analog of claim 1, wherein the B-chain N-terminus, the B-chain C-terminus, or both the B-chain N-terminus and the B-chain C-terminus is substituted with a substituent.

3. The insulin analog of claim 1, wherein the substituent is a AEEA-AEEA-γ-Glu-(CH2)ₘ—OH, wherein m is 12, 14, 16, 18, 20, or 22.

4. The insulin analog of claim 1, wherein A6 and A11 are connected by a linker, A7 and B7 are connected by a linker, A20 and B19 are connected by a linker, or a combination thereof.

5. (canceled)

6. (canceled)

7. The insulin analog of claim 4, wherein the linker is cleavable or non-cleavable.

8. The insulin analog of claim 4, wherein the linker comprises a disulfide bond.

9. The insulin analog of claim 1, wherein the insulin analog comprises a binding affinity (EC₅₀) to insulin receptor is below 10 nM.

10. (canceled)

11. (canceled)

12. The insulin analog of claim 1, wherein an insulin analog binding affinity is measured by surface plasmon resonance, by using a recombinant cell line, or a combination thereof, wherein the recombinant cell line expresses an insulin receptor.

13. (canceled)

14. (canceled)

15. The insulin analog of claim 12, wherein the insulin analog binding affinity (IC₅₀) to the insulin receptor is determined by measuring luciferase activity.

16. The insulin analog of claim 1, wherein the insulin analog exhibits an in vivo plasma elimination half-life of at least 1 hour, at least 2 hours, at least 4 hours, at least 6 hours, at least 8 hours, at least 10 hours, at least 24 hours, at least 48 hours, at least 72 hours, or at least 168 hours in humans.

17. A pharmaceutical composition comprising the insulin analog of claim 1 and a pharmaceutically acceptable vehicle or carrier.

18. The pharmaceutical composition of claim 17, further comprising an isotonic agent, an excipient, a preservative, a buffer, a surfactant, an anti-diabetic agent, or a combination thereof.

19. (canceled)

20. (canceled)

21. The pharmaceutical composition of claim 17, wherein the pharmaceutically acceptable carrier is a microsphere, a micelle, or a nanoparticle.

22. A method of treating diabetes, comprising administering to a patient a therapeutically effective amount of the pharmaceutical composition of claim 17.

23. The method of claim 22, wherein the treatment results in a reduction or stabilization of average blood glucose levels in the patient.

24. The method of claim 22, wherein the pharmaceutical composition is administered by oral, subcutaneous, intravenous, parenteral, transdermal, intramuscular, rectal, vaginal, pump-based, or topical administration.

25. (canceled)

26. The method of claim 22, wherein the pharmaceutical composition is administered once weekly.

27. The method of claim 22, wherein administration of the pharmaceutical composition provides a longer lasting glucose lowering effect, a longer lasting c-peptide lowering effect, a lower body weight increase, or a combination thereof compared to administration of degludec.

28. (canceled)

29. (canceled)

30. The method of claim 22, wherein the administration of the pharmaceutical composition does not activate the insulin-like growth factor-1 receptor (IGF1R).
